SEQUENCE LISTING (5 sequences)

SEQ ID NO: 1 - DNA, 20 bp, Artificial Sequence
  Description of Artificial Sequence Synthetic primer
gaacattgcctatggagaca

SEQ ID NO: 2 - DNA, 20 bp, Artificial Sequence
  Description of Artificial Sequence Synthetic primer
ccaggctgtttatttgaaga

SEQ ID NO: 3 - DNA, 52 bp, Artificial Sequence
  Description of Artificial Sequence Synthetic primer
ttttttttttccccccccccccaggctgtttatttgaagagagacttacatt

SEQ ID NO: 4 - DNA, 20 bp, Artificial Sequence
  Description of Artificial Sequence Synthetic oligonucleotide
ttttttttttcccccccccc

SEQ ID NO: 5 - DNA, 132 bp, Artificial Sequence
  Description of Artificial Sequence Synthetic polynucleotide
gaacattgcctatggagacaacagccgggtggtgtcacaggaagagatcgtgagggcagcaaaggaggccaacatacatgccttcatcgagtcactgcctaatgtaagtctctcttcaaataaacagcctgg

(19) United States
(12) Patent Application Publication
(10) Pub. No.: US 2019/0178804 A1
(43) Pub. Date: Jun. 13, 2019

(54) LIGHT-ABSORBING OPTICAL FIBER-BASED SYSTEMS AND METHODS

(71) Applicant: Spartan Bioscience Inc., Ottawa (CA)

(72) Inventors: Hubert Jean-Ruel, Ottawa (CA); Jacques Albert, Gatineau (CA); Jason Koppert, Toronto (CA); Chris Harder, Dunrobin (CA); Sergey Golovan, Ottawa (CA); Alan Shayanpour, Stittsville (CA); Anatoli Ianoul, Nepean (CA); Jason Coyle, Ottawa (CA)

(21) Appl. No.: 16/324,491

(22) PCT Filed: Aug. 2, 2017
(86) PCT No.: PCT/CA17/50925

§ 371 Date: Feb. 8, 2019

Related U.S. Application Data
(60) Provisional application No. 62/374,506, filed on Aug. 12, 2016.

(51) Int. Cl. G01N 21/77; C12Q 1/6825; G02B 6/42

(52) U.S. Cl. CPC G01N 21/7703; C12Q 1/6825; G01N 2021/7786; G02B 6/4215; G01N 2021/7716; G02B 6/4206

(57) ABSTRACT

The present disclosure relates to optical fiber-based devices, and more particularly to light-absorbing optical fiber-based systems and methods.

### CROSS-REFERENCE TO RELATED APPLICATION

This application claims the benefit of U.S. Provisional Application Ser. No. 62/374,506 filed on Aug. 12, 2016, the disclosure of which is hereby incorporated by reference.

### SEQUENCE LISTING

The present specification makes reference to a Sequence Listing (submitted electronically as a .txt file named “2009379-0040_SL.txt” on Aug. 2, 2017). The .txt file was generated on Jul. 20, 2017 and is 1,566 bytes in size. The entire contents of the Sequence Listing are hereby incorporated by reference.

### FIELD

The present disclosure relates to optical fiber-based devices, and more particularly to light-absorbing optical fiber-based systems and methods.

### BACKGROUND

Although a variety of systems and methods for performing PCR or quantitative PCR (qPCR) are known in the art, current methods and systems are typically limited in their multiplex capacity (current methods and systems are generally only able to detect two or three different targets). This limitation is attributed to the spectral overlap among the emission spectra of fluorescent reporters attached to oligonucleotides that are used to detect targets. To overcome this issue, Daems et al. (2016) developed a Fiber Optic Surface Plasmon Resonance (FO-SPR) biosensor with hybridization probes immobilized on the optical fiber surface (Daems D et al., Real-time PCR melting analysis with fiber optic SPR enables multiplex DNA identification of bacteria. Analyst. 141:1906-1911, 2016). However, one of the drawbacks of this device is that a separate thermal cycler is required to manipulate and measure the sample temperature. There remains a need for optical fiber-based devices which are capable of multiplex detection of analytes in a sample (e.g., by qPCR) yet do not require a separate thermal cycler to manipulate and measure the sample temperature.

### SUMMARY

In some embodiments, systems and methods discussed herein provide optical fiber-based devices that enable multiplexed detection of analytes in a sample without the need for a separate thermal cycler to manipulate and measure the sample temperature. In some embodiments, these systems and methods provide temperature control (e.g., the ability to increase, decrease, and/or maintain the temperature of the sample) and measurement of analytes in a sample using an efficient and selective light-coupling scheme between the instrument (e.g., wherein a scheme includes, among other things, multiple light sources) and the device. In some embodiments, the device functionality results from certain optical fiber design features including a first region (e.g., a doped core) that transmits and progressively absorbs infrared light, a second region (e.g., a cladding that surrounds a doped core) that propagates visible light and enables evanescent field excitation of its surface, and biomolecules attached to the surface of the second region (e.g., cladding) that fluoresce when excited by the evanescent field. In some embodiments, the multiplex capabilities of the device results from spatial separation of the detection signals associated with each analyte measured within a single reaction vessel. The present disclosure provides various configurations of optical fiber-based systems and methods that can be used for multiplexed detection of analytes without a separate thermal cycler.

In one aspect, the present disclosure provides systems comprising: a light-absorbing optical fiber (LAF), which includes a first region and a second region, wherein the first region absorbs light at a first wavelength and the second region transmits light at a second wavelength and wherein the first and second regions both extend along the entire length of the LAF and each have longitudinal axes that are parallel to a longitudinal axis through the center of the LAF; a first light source that produces light having the first wavelength and a second light source that produces light having the second wavelength; and an optical coupling element configured to (i) couple light having the first wavelength from the first light source into the first region of the LAF and (ii) couple light having the second wavelength from the second light source into the second region of the LAF.

In some embodiments, the first wavelength is in the infrared region, e.g., from about 750 nm to 50 um (e.g., from about 1300 to 1700 nm, e.g., from about 1552 to 1553 nm). In some embodiments, the first region absorbs light at the first wavelength with an attenuation in the range of about 0.1 dB/cm to 10 dB/cm (e.g., in the range of about 1.5 dB/cm to 2.5 dB/cm). In some embodiments, the first region includes a dopant selected from the group consisting of the transition elements Co, Fe, Ni, Cr, Cu, Ti, Mn, V, and combinations thereof.

In some embodiments, the second wavelength is in the region from about 390 nm to 700 nm (e.g., from about 450 nm to 500 nm, e.g., about 488 nm).

In some embodiments, the first and second regions are in physical contact (e.g., wherein first region is a core region of the LAF and the second region is a cladding layer surrounding the core region of the LAF).

In some embodiments, the LAF is about 2 mm to 50 mm in length (e.g., about 5 mm to 20 mm in length). In some embodiments, the first region is a core region of the LAF and the second region is a cladding layer surrounding the core region of the LAF, and wherein the diameter of the core region of the LAF is in the range of about 6μm to 1000 μm (e.g., about 7 μm to 50 μm).

In some embodiments, power output of the first light source at the first wavelength is variable. In some embodiments, the first light source is a variable amplifier (e.g., erbium-doped fiber amplifier (EDFA) seeded with a tunable fiber laser adjusted to the first wavelength). In some embodiments, the first light source is a high power laser diode tuned to the first wavelength and controlled with a laser diode driver which has an adjustable current output.

In some embodiments, power output of the first light source at the first wavelength can be varied from 0 Watt up to about 20 Watt (e.g., from 0 Watt up to about 2 Watt).

In some embodiments, the second light source includes a laser upstream of an optical shutter and a filter that transmits only the second wavelength. In some embodiments, the second light source includes a fiber pigtailed laser diode with an output centered at the second wavelength. In some embodiments, a laser diode driver is used to enable/disable the output of the fiber pigtailed laser diode.

In some embodiments, the optical coupling element comprises a free space coupler. In some embodiments, the provided systems comprise an optical fiber element upstream of the free space coupler in which the first and second light sources are multiplexed.

In some embodiments, the provided systems comprise a low-loss optical fiber which comprises a core region and a cladding layer surrounding the core region, and wherein the first and second wavelengths are multiplexed in the core region of the low-loss optical fiber.

In some embodiments, the optical coupling element comprises a multimode interference (MMI) element spliced upstream of the LAF. In some embodiments, the MMI element comprises a low-loss single mode optical fiber element spliced upstream of a low-loss multimode optical fiber element, and wherein the first and second wavelengths are multiplexed in the core of the low-loss single mode optical fiber element. In some embodiments, the core of the low-loss multimode optical fiber is larger than the core of the LAF.

In some embodiments, the LAF-based device comprises a low-loss optical fiber element spliced upstream of the LAF. In some embodiments, the optical fiber element is a low-loss optical fiber which comprises a core region and a cladding layer surrounding the core region (e.g., a single cladding low-loss optical fiber, e.g., a double cladding low-loss optical fiber).

In some embodiments, the first wavelength is transmitted through the core region of the low-loss optical fiber and the second wavelength is transmitted through a cladding layer of the low-loss optical fiber.

In some embodiments, the provided systems further comprise a reflective element (e.g., a mirror, e.g., a gold mirror, e.g., a chirped fiber grating, e.g., a chirped fiber grating which reflects light in the range from about 1551 nm to 1554 nm) located downstream of the LAF, wherein at least a portion of the light at the first wavelength is reflected back into the LAF by the reflective element (e.g., wherein the reflective element has a reflectivity greater than 80%, e.g., 90%, e.g., 95%) (e.g., wherein the combination of the first light pass and the reflected pass results in a relative difference in absorbed power (per unit length) between the downstream and upstream ends (e.g., a relative difference below 50%) and results in a total attenuation in the core of the LAF ranging from about 0.5 dB to 5.5 dB). In some embodiments, the reflective element is a chirped fiber grating that has a reflection spectrum that both transmits the first wavelength at an annealing and/or extension temperature and reflects the first wavelength at a denaturation temperature.

In some embodiments, the provided systems further comprise a Fiber Bragg Grating (FBG) inscribed within the first region of the LAF or within a low-loss optical fiber spliced upstream of the LAF.

In some embodiments, the provided systems further comprise a third light source that produces light covering a range of wavelengths (e.g., in the infrared region) for interrogating the FBG and an optical spectrum analyzer for monitoring the Bragg peak of the FBG. In some embodiments, the provided systems further comprise a third light source that produces light having a third wavelength; and a power meter (or photodiode) with a bandpass filter for monitoring the reflected power of this third light source to infer the spectral position of the Bragg peak.

In some embodiments, the light produced by the third light source is in the infrared region, e.g., from about 750 nm to 3 μm (e.g., from about 1525 nm to 1610 nm, e.g., about 1567 nm). In some embodiments, the third light source is a broadband infrared light source.

In some embodiments, power output of the third light source at the third wavelength or range of wavelengths is less than about 0.1 Watt (e.g., less than about 0.01 Watt).

In some embodiments, the provided systems further comprise a detection element (e.g., an imaging element) for detecting (e.g., imaging) fluorescence on an outside surface of the second region of the LAF (e.g., a camera, e.g., a camera with lenses and an emission filter, e.g., a 515 nm to 540 nm emission filter).

In some embodiments, the provided systems further comprise a support structure (e.g., a ferrule) in contact with the LAF or upstream low-loss fiber section. In some embodiments, the provided systems further comprise a reaction vessel, wherein at least a portion of the LAF is located within the reaction vessel (e.g., an open ended glass capillary). In some embodiments, the reaction vessel includes a glass capillary closed on one side by a ferrule containing the LAF and closed on the other side by a cap.

In some embodiments, the provided systems further comprise an immobilized (e.g., non-covalently, e.g., covalently) capture probe for an analyte on an outside surface of the second region of the LAF. In some embodiments, the immobilized capture probe comprises a biomolecule selected from the group consisting of polynucleotides (e.g., RNA, DNA, PNA, oligonucleotide that are at least partially complementary to the analyte), polypeptides (e.g., antibodies or receptors that bind the analyte), and polysaccharides (e.g., that are bound by the analyte).

In some embodiments, a plurality of one or more types of capture probes specific for one or more types of analytes is immobilized in an array format on an outside surface of the second region of the LAF.

In some embodiments, the provided systems further comprise a nanoparticle coating on an outside surface of the second region of the LAF.

In some embodiments, the provided systems further comprise an external heating and/or cooling element (e.g., wherein the element includes a member selected from the group consisting of a fan, an element that includes a liquid coolant, cooled and/or heated air streams, a Peltier module, a resistive heater, and combinations thereof).

In an additional aspect, the present disclosure provides methods comprising: providing any of the systems described herein, wherein the reaction vessel includes a liquid sample; and transmitting light having the first wavelength from the first light source into the first region of the LAF to heat the liquid sample.

In some embodiments, the provided methods further comprise transmitting light having the second wavelength from the second light source into the second region of the LAF.

In some embodiments, a Fiber Bragg Grating (FBG) is inscribed within the first region of the LAF or within a low-loss optical fiber spliced upstream of the LAF and the provided systems comprise a third light source that produces light for interrogating the FBG and an optical spectrum analyzer for monitoring the Bragg peak of the FBG, the method further comprising transmitting light from the third light source; and monitoring the Bragg peak of the FBG using the optical spectrum analyzer.

In some embodiments, the provided methods further comprise detecting (e.g., imaging) (e.g., via a detection element (e.g., an imaging element)) fluorescence on an outside surface of the second region of the LAF (e.g., using a camera, e.g., a camera with lenses and an emission filter, e.g., a 515 nm to 540 nm emission filter), wherein the fluorescence is indicative of the presence of an analyte in the liquid sample. In some embodiments, the liquid sample comprises a nucleic acid analyte and amplification reagents (e.g., forward and reverse primers that hybridize to the nucleic acid analyte and its complement and a DNA polymerase) (e.g., wherein the liquid sample is a biological sample).

In some embodiments, forward and reverse primers are used with non-equal concentrations (e.g., at a ratio of from about 1:2 to about 1:4, e.g., at a ratio of about 1:3, e.g., at a ratio of from about 1:8 to about 1:12).

In some embodiments, the provided methods further comprise an immobilized (e.g., non-covalently, e.g., covalently) capture probe for an analyte on an outside surface of the second region of the LAF (e.g., wherein the immobilized capture probe is a forward or reverse primer for amplification of a nucleic acid analyte, e.g., wherein the immobilized capture probe is an oligonucleotide comprising a section complementary to at least a portion of the analyte). In some embodiments, the immobilized capture probe comprises an oligonucleotide that hybridizes to a nucleic acid analyte. In some embodiments, the liquid sample comprises a nucleic acid analyte. In some embodiments, the liquid sample further comprises a fluorescent reporter that preferentially binds to double-stranded nucleic acid molecules over single-stranded nucleic acid molecules (e.g., double-stranded DNA over single-stranded DNA) and absorbs light at the second wavelength (e.g., an intercalating dye, e.g., SYBR green). In some embodiments, the liquid sample further comprises a labeled (e.g., with a fluorophore) oligonucleotide detection probe that directly or indirectly hybridizes to the nucleic acid analyte. In some embodiments, the oligonucleotide detection probe is a molecular beacon detection probe.

In some embodiments, a plurality of different molecular beacons are used to detect a plurality of different nucleic acid analytes in the liquid sample.

In some embodiments, the provided methods further comprise adjusting the output power of the first light source (e.g., in the range of 0 Watt up to about 20 Watt, e.g., in the range from 0 Watt up to about 2 Watt) to cycle a temperature of the liquid sample. In some embodiments, the step of adjusting leads to PCR amplification of a nucleic acid analyte in the liquid sample.

In some embodiments, the PCR amplification involves extension of at least one forward and/or reverse primer that is immobilized on the surface of the second region of the LAF.

In some embodiments, the step of adjusting increases the temperature of the liquid sample. In some embodiments, the step of adjusting reduces the temperature of the liquid sample.

In some embodiments, the provided methods further comprise controlling the output power of the first light source to maintain a temperature of the liquid sample. In some embodiments, the step of controlling leads to isothermal amplification of a nucleic acid analyte in the liquid sample. In some embodiments, the isothermal amplification involves extension of at least one forward and/or reverse primer that is immobilized on the surface of the second region of the LAF.

In some embodiments, the provided methods further comprise heating and/or cooling the provided systems via a heating and/or cooling element (e.g., wherein the heating and/or cooling element includes a member selected from the group consisting of a fan, an element that includes a liquid coolant, cooled and/or heated air streams, a Peltier module, a resistive heater, and combinations thereof) (e.g., wherein the heating and/or cooling is achieved by using a combination of the heating and/or cooling elements and IR light).

In some embodiments, the provided methods further comprise monitoring temperature via a member selected from the group consisting of a thermistor, a thermocouple, an RTD, and a non-contact IR thermometer (e.g., wherein the temperature is also monitored by the systems provided herein).

In some embodiments, an external (not fiber-coupled) light source is used (e.g., to monitor fluorescence in the liquid sample).

### BRIEF DESCRIPTION OF THE DRAWING

Drawings are presented herein for illustration purposes, not for limitation.

The foregoing and other objects, aspects, features, and advantages of the present disclosure will become more apparent and better understood by referring to the following description taken in conjunction with the accompanying drawing, in which:

FIG. 1 depicts an exemplary light-absorbing optical fiber (LAF)-based device (1). The legend for the highlighted components is as follows: (1) LAF-based device, (2) Standard Low-Loss fiber, (4) Light-Absorbing Fiber (LAF), (5) Fiber core, (6) Fiber cladding, (7) Dopant, (8) Fiber Bragg Grating (FBG) for temperature measurement, (9) Chirped FBG for reflecting light, (10) Visible light, (11) Infra-red light, (12) Biomolecules attached to cladding surface.

FIG. 2 depicts an exemplary LAF-based device in which a Free space coupler (13) is used to couple multiplexed light (10, 11) from a Standard Low-Loss Fiber (2) into the LAF (4) in a wavelength-selective way. The legend for the highlighted components is as follows: (2) Standard Low-Loss Fiber, (4) Light-Absorbing Fiber (LAF), (5) Fiber core, (6) Fiber cladding, (7) Dopant, (10) Visible light, (11) Infrared light, (13) Free space coupler.

FIG. 3 shows an exemplary configuration of a LAF-based device (1). The legend for the highlighted components is as follows: (1) LAF-based device, (4) Light-Absorbing Fiber (LAF), (14) Ferrule, (15) Capillary, (16) Capillary cap, (17) Standard or double-cladding low-loss fiber.

FIG. 4 shows an exemplary configuration of a LAF-based device where a multimode interference (MMI) section is used for completely in-fiber coupling. The legend for the highlighted components is as follows: (2) Single Mode Fiber (SMF), (3) Multimode Fiber, (4) Light-Absorbing Fiber (LAF), (5) Fiber core, (6) Fiber cladding, (7) Dopant, (10) Visible light, (11) Infrared light.

FIG. 5 is a block diagram of an exemplary network environment for use in the methods and systems for analysis of spectrometry data, according to an illustrative embodiment.

FIG. 6 is a block diagram of an exemplary computing device and an exemplary mobile computing device, for use in illustrative embodiments of the invention.

FIG. 7 shows real-time monitoring of the fluorescence of a DNA spot during qPCR with an exemplary LAF-based device. The spot intensity is normalized to the background intensity of the fiber at the first cycle. A sigmoidal fit is also displayed.

FIG. 8 shows fluorescence intensity (integrated over the width of the fiber) along the longitudinal axis of the LAF section before and after PCR amplification. Successful amplification of the three DNA spots is unambiguously observed.

FIG. 9 shows a melt curve of a DNA spot acquired in situ with an exemplary LAF-based device following PCR. The spot intensity is normalized to its fluorescence intensity at 59° C. A melt temperature of approximately 82° C. is observed, which agrees well with the expected value based on calculations.

### DEFINITIONS

Throughout the specification, several terms are employed that are defined in the following paragraphs. Other definitions are also found within the body of the specification.

As used herein, the terms “about” and “approximately,” in reference to a number, are used to include numbers that fall within a range of 20%, 10%, 5%, or 1% in either direction (greater than or less than) of the number unless otherwise stated or otherwise evident from the context (except where such number would exceed 100% of a possible value).

As used herein, the term “amplification,” when used in reference to polynucleotides, refers to a method that increases the representation in a population of a specific nucleotide sequence (e.g., from a template polynucleotide) in a sample by producing multiple (i.e., at least 2) copies of the desired nucleotide sequence. Methods for nucleic acid amplification are known in the art and include, but are not limited to, polymerase chain reaction (PCR) and ligase chain reaction (LCR). Variants of standard PCR or LCR reactions can also be used. A “copy” or “amplicon” does not necessarily have perfect sequence complementarity or identity to the nucleotide sequence in the template polynucleotide. Unless otherwise specified, one or more copies can comprise one or more mutant copies, i.e., copies containing one or more mutations (“mutant copies”) as compared to the nucleotide sequence in the template polynucleotide. Mutant copies can comprise mutations in one or more bases. For example, for template polynucleotides that comprise a coding region with a plurality of codons, mutant copies can comprise mutations in one or more than one codon and within each codon, there can be mutations in one, two, or all three nucleotides of the codon. In general, “mutations” will be understood to include substitutions, insertions or deletions relative to the template polynucleotide. In some embodiments, amplification is performed by adjusting a temperature of a sample (i.e., thermocycling). In some embodiments, amplification is performed by maintaining a temperature of a sample constant (i.e., isothermal).

As used herein, the term “analyte,” consistent with its use in the art, refers to an entity whose presence, level, or form, correlates with a particular biological event or state of interest, so that it is considered to be a “marker” of that event or state. To give but a few examples, in some embodiments, an analyte may be or comprise a marker for a particular disease state, or for likelihood that a particular disease, disorder or condition may develop, occur, or reoccur. In some embodiments, an analyte may be or comprise a marker for a particular disease or therapeutic outcome, or likelihood thereof Thus, in some embodiments, an analyte is predictive, in some embodiments, an analyte is prognostic, in some embodiments, an analyte is diagnostic, of the relevant biological event or state of interest. An analyte may be an entity of any chemical class. For example, in some embodiments, an analyte may be or comprise a nucleic acid, a polypeptide, a lipid, a carbohydrate, or a combination thereof In some embodiments, an analyte is a cell surface marker. In some embodiments, an analyte is intracellular. In some embodiments, an analyte is found outside of cells (e.g., is secreted or is otherwise generated or present outside of cells, e.g., in a body fluid such as blood, urine, tears, saliva, cerebrospinal fluid, etc.).

As used herein, the term “array” refers to a population of different molecules (e.g., capture probes) that are attached to one or more substrates such that the different molecules can be differentiated from each other according to relative location. An array can include different molecules that are each located at a different addressable location on a substrate. Alternatively, an array can include separate substrates each bearing a different molecule. Different molecules associated with separate substrates can be identified according to the locations of the substrates on a surface to which the substrates are associated or according to the locations of the substrates in a sample.

As used herein, the term “antibody” refers to an immunoglobulin which specifically binds to and is thereby defined as complementary with a particular spatial and polar organization of another molecule, including recombinant antibodies such as chimeric antibodies and humanized antibodies. The antibody can be monoclonal or polyclonal and can be prepared by techniques that are well known in the art such as immunization of a host and collection of sera (polyclonal) or by preparing continuous hybrid cell lines and collecting the secreted protein (monoclonal), or by cloning and expressing nucleotide sequences or mutagenized versions thereof coding at least for the amino acid sequences required for specific binding of natural antibodies. Antibodies may include a complete immunoglobulin or fragment thereof, which immunoglobulins include the various classes and isotypes, such as IgA, IgD, IgE, IgG1, IgG2a, IgG2b and IgG3, IgM, etc. Fragments thereof may include Fab, Fv and F(ab′)₂, Fab′, and the like. In addition, aggregates, polymers, and conjugates of immunoglobulins or their fragments can be used where appropriate so long as binding affinity for a particular molecule is maintained.

As used herein, the term “capture probe” refers to a molecule (e.g., a biomolecule) capable of binding to a target analyte, e.g., capable of hybridizing to a target nucleic acid.

As used herein, the term “hybridize” or “hybridization” refers to a process where two strands in a double-stranded polynucleotide anneal to each other under appropriately stringent conditions. The phrase “is capable is hybridizing to” refers to the ability of two strands of double-stranded polynucleotide to hybridize to each other under typical hybridization conditions (e.g., in the context of a typical amplification reaction, “hybridize” would refer to the interaction of two complementary nucleotide sequences during the annealing phase). As understood by one of ordinary skill in the art, nucleotide sequences need not have perfect sequence complementarity to hybridize with one another. Those skilled in the art understand how to estimate and adjust the stringency of hybridization conditions such that sequences having at least a desired level of complementary will stably hybridize, while those having lower complementary will not. For examples of hybridization conditions and parameters, see, e.g., Sambrook, et al., 1989, Molecular Cloning: A Laboratory Manual, Second Edition, Cold Spring Harbor Press, Plainview, N.Y.; Ausubel, et al. 1994, Current Protocols in Molecular Biology. John Wiley & Sons, Secaucus, N.J.

As used herein, the term “immobilized” or “attached” can include direct or indirect association with a substrate via covalent and/or non-covalent bonds. In some embodiments, covalent attachment may be used, but generally all that is required is that the molecules (e.g., biomolecules such as nucleic acids) remain immobilized or attached to a support under conditions in which it is intended to use the substrate, for example in applications requiring nucleic acid amplification and/or sequencing. Typically oligonucleotides to be used as capture oligonucleotides or amplification oligonucleotides are immobilized such that a 3′ end is available for enzymatic extension and at least a portion of the sequence is capable of hybridizing to a complementary sequence. Immobilization can occur via hybridization to a surface attached oligonucleotide, in which case the immobilized oligonucleotide or polynucleotide may be in the 3′-5′ orientation. Alternatively, immobilization can occur by means other than base-pairing hybridization, such as the covalent attachment. There are a wide variety of known methods of attaching nucleic acids to substrates that include attachment of binding ligands, including nucleic acid probes, to microspheres that are randomly distributed on a surface, including an optical fiber bundle, to form high density arrays. See for example PCT Publication Nos. WO/1999/018434, WO/1998/040726, WO/1998/040726, and WO/1998/050782; all of which are expressly incorporated herein by reference in their entireties.

As used herein, the term “labeled” refers to incorporation of a detectable marker, e.g., by incorporation of a fluorophore. Various types of detectable markers (e.g., fluorophores) that can be used include, but are not limited to, those that are described herein.

As used herein, the terms “nucleic acid”, “nucleic acid molecule” or “polynucleotide” are used herein interchangeably. They refer to a polymer of deoxyribonucleotides or ribonucleotides in either single- or double-stranded form, and unless otherwise stated, encompass known analogs of natural nucleotides that can function in a similar manner as naturally occurring nucleotides. The terms encompass nucleic acid-like structures with synthetic backbones, as well as amplification products. DNAs and RNAs are both polynucleotides. The polymer may include natural nucleosides (i.e., adenosine, thymidine, guanosine, cytidine, uridine, deoxyadenosine, deoxythymidine, deoxyguanosine, and deoxycytidine), nucleoside analogs (e.g., 2-aminoadenosine, 2-thiothymidine, inosine, pyrrolo-pyrimidine, 3-methyl adenosine, C5-propynylcytidine, C5-propynyluridine, C5-bromouridine, C5-fluorouridine, C5-iodouridine, C5-methylcytidine, 7-deazaadenosine, 7-deazaguanosine, 8-oxoadenosine, 8-oxoguanosine, O(6)-methylguanine, and 2-thiocytidine), chemically modified bases, biologically modified bases (e.g., methylated bases), intercalated bases, modified sugars (e.g., 2′-fluororibose, ribose, 2′-deoxyribose, arabinose, and hexose), or modified phosphate groups (e.g., phosphorothioates and 5′-N-phosphoramidite linkages).

As used herein, the term “oligonucleotide” refers to a polynucleotide. Oligonucleotides may be obtained by a number of methods including, for example, chemical synthesis, restriction enzyme digestion or PCR. As will be appreciated by one skilled in the art, the length of an oligonucleotide (i.e., the number of nucleotides) can vary widely, often depending on the intended function or use of the oligonucleotide. In some embodiments, oligonucleotides comprise between about 5 and about 300 nucleotides, for example, between about 15 and about 200 nucleotides, between about 15 and about 100 nucleotides, between about 15 and about 50 nucleotides, and between about 20 and about 40 nucleotides. In some embodiments, oligonucleotides are between about 20 and about 40 nucleotides in length.

As used herein, the term “plurality” means more than one.

As used herein, the term “polypeptide” generally has its art-recognized meaning of a polymer of amino acids. The term is also used to refer to specific functional classes of polypeptides, such as, for example, nucleases, antibodies, etc.

As used herein, the term “primer” is interchangeable with “oligonucleotide primer” and is used herein to refer to an oligonucleotide that acts as a point of initiation of synthesis of a primer extension product when hybridized to a template polynucleotide, when placed under suitable conditions (e.g., buffer, salt, temperature and pH), in the presence of amplification reagents (e.g., nucleotides and an agent for nucleic acid polymerization, e.g., a DNA-dependent or RNA-dependent polymerase). The primer is preferably single-stranded for maximum efficiency in amplification, but may alternatively be double-stranded. If double-stranded, the primer may first be treated (e.g., denatured) to allow separation of its strands before being used to prepare extension products. Such a denaturation step is typically performed using heat, but may alternatively be carried out using alkali, followed by neutralization. A typical primer comprises a sequence of about 10 to about 50, e.g., about 20 to about 40 nucleotides that is complementary to a sequence in a template polynucleotide.

As used herein, the term “sample” typically refers to a biological sample obtained or derived from a source of interest, as described herein. In some embodiments, a source of interest comprises an organism, such as a microbe, a plant, an animal or a human. In some embodiments, a biological sample is or comprises biological tissue or fluid. In some embodiments, a biological sample may be or comprise bone marrow; blood; blood cells; ascites; tissue or fine needle biopsy samples; cell-containing body fluids; free floating nucleic acids (e.g., cell free DNA); sputum; saliva; urine; cerebrospinal fluid, peritoneal fluid; pleural fluid; lymph; gynecological fluids; skin swabs; vaginal swabs; oral swabs; nasal swabs; washings or lavages such as a ductal lavages or broncheoalveolar lavages; aspirates; scrapings; bone marrow specimens; tissue biopsy specimens; surgical specimens; feces, other body fluids, secretions, and/or excretions; and/or cells therefrom, etc. In some embodiments, a biological sample is or comprises cells obtained from an individual. In some embodiments, obtained cells are or include cells from an individual from whom the sample is obtained. In some embodiments, a sample is a “primary sample” obtained directly from a source of interest by any appropriate means. For example, in some embodiments, a primary biological sample is obtained by methods selected from the group consisting of a swab, biopsy (e.g., fine needle aspiration or tissue biopsy), surgery, collection of body fluid (e.g., blood, lymph, feces etc.), etc. In some embodiments, as will be clear from context, the term “sample” refers to a preparation that is obtained by processing (e.g., by removing one or more components of and/or by adding one or more agents to) a primary sample. For example, filtering using a semi-permeable membrane. Such a processed “sample” may comprise, for example nucleic acids or proteins extracted from a sample or obtained by subjecting a primary sample to techniques such as amplification or reverse transcription of mRNA, isolation and/or purification of certain components, etc.

As used herein, the term “specific”, when used herein with reference to an agent having an activity, is understood by those skilled in the art to mean that the agent discriminates between potential target entities or states. For example, in some embodiments, an agent is said to bind “specifically” to its target if it binds preferentially with that target in the presence of one or more competing alternative targets. In many embodiments, specific interaction is dependent upon the presence of a particular structural feature of the target entity (e.g., a nucleotide sequence, an epitope, a cleft, a binding site). It is to be understood that specificity need not be absolute. In some embodiments, specificity may be evaluated relative to that of the binding agent for one or more other potential target entities (e.g., competitors). In some embodiments, specificity is evaluated relative to that of a reference specific binding agent. In some embodiments specificity is evaluated relative to that of a reference non-specific binding agent. In some embodiments, the agent does not detectably bind to the competing alternative target(s) under conditions of binding to its target entity. In some embodiments, the agent binds with higher on-rate, lower off-rate, increased affinity, decreased dissociation, and/or increased stability to its target entity as compared with the competing alternative target(s).

As used herein, the term “sensor” includes any sensor of electromagnetic radiation including, but not limited to, CCD camera, photomultiplier tubes, photodiodes, and avalanche photodiodes, unless otherwise evident from the context.

### DETAILED DESCRIPTION OF SOME EMBODIMENTS

Throughout the description, where systems or devices are described as having, including, or comprising specific components, or where methods are described as having, including, or comprising specific steps, it is contemplated that, additionally, there are systems or devices of the present invention that consist essentially of, or consist of, the recited components, and that there are methods according to the present invention that consist essentially of, or consist of, the recited processing steps.

It should be understood that the order of steps or order for performing certain actions is immaterial so long as the invention remains operable. Moreover, two or more steps or actions may be conducted simultaneously.

The mention herein of any publication, for example, in the Background section, is not an admission that the publication serves as prior art with respect to any of the claims presented herein. The Background section is presented solely for purposes of clarity and is not meant as a description of prior art with respect to any claim.

The present invention encompasses the recognition that an optical fiber-based device capable of multiplex detection, thermal cycling, and temperature measurement is desired. In some embodiments, such an optical fiber-based device includes a first region (e.g., a doped core) that transmits and progressively absorbs infrared (IR) light, a second region (e.g., a cladding that surrounds a doped core) that propagates visible light and enables evanescent field excitation of its surface, and biomolecules attached to the surface of the second region (e.g., cladding) that fluoresce when excited by the evanescent field. Various embodiments that possess these features are described in detail herein. The described embodiments are not intended to be limiting.

### I. First Region (e.g., Doped Core) That Transmits and Progressively Absorbs Infrared Light

In some embodiments, the first region (e.g., core) is doped so that it absorbs infrared (IR) light transmitting through it. Examples of fibers that can be used in the presented systems and methods include, but are not limited to, an ATN-FB fiber (e.g., attenuation range=0.4 dB/cm-15 dB/cm) available from CorActive (Quebec, Canada): coractive.com/products/passive-fibers/attenuating/index.html, or equivalent (e.g., custom-made fiber, e.g., commercially available) attenuation fibers.

Temperature Control Using IR Pump

As IR light is transmitted through the first region (e.g., doped core) of the fiber, the IR light is progressively absorbed along its length and converted into heat. The entire fiber section and its surrounding environment are heated via conduction, and a steady-state temperature depending on the input power of the IR pump is reached. In some embodiments, the input power of the IR pump is increased to increase the temperature of the first region (e.g., doped core) and surrounding fiber for thermal cycling (e.g., for PCR). In some embodiments, the input power of the IP pump is decreased to decrease the temperature of the first region (e.g., doped core) and surrounding fiber for thermal cycling (e.g., for PCR). In some embodiments, the IR pump is increased and/or decreased so that a temperature of the first region (e.g., doped core) and surrounding fiber is maintained for isothermal reactions. In some embodiments, a system of the present disclosure may include an external cooling/and or heating element (e.g., wherein the element includes a member selected from the group consisting of a fan, an element that includes a liquid coolant, cooled and/or heated air streams, a Peltier module, and a resistive heater, and combinations thereof) that is used in conjunction with IR light to adjust, maintain, or cycle the temperature of the first region (e.g., doped core) and surrounding fiber.

Dopant Concentration in the First Region (e.g., Doped Core) of the Light-Absorbing Fiber (LAF)-Based Device

As described herein, the dopant concentration in the first region (e.g., doped core) of the LAF-based device is generally selected to provide optimal heating efficiency (e.g., to lower the IR pump power—and thus cost—requirement) while providing the desired temperature and heating profile uniformity with respect to the axial absorption profile. For example, the first region's (e.g., doped core) absorptivity at the IR pump wavelength cannot be made arbitrarily large with the goal of providing maximal heating efficiency since the pump light would then be rapidly attenuated upon its entrance into the first region (e.g., doped core) and thus only the upstream side would be heated. Conversely, if the first region's (e.g., doped core) absorptivity is made arbitrarily low such as to minimize depletion along the first region's (e.g., doped core) axis, the absorption profile will be uniform but the overall heating efficiency will be correspondingly low.

Considering the above, the maximum attenuation A (in dB/cm) that one of ordinary skill in the art can select for a segment of length L (in cm) is limited by the maximum acceptable relative difference ≢X in absorbed power (per unit length) at the downstream end of the first region (e.g., doped core) with respect to the upstream end. This maximum acceptable AX depends on the reaction vessel geometry and materials, the environmental condition, and the specific reaction (e.g., PCR reaction).

For a single pump pass embodiment (no downstream reflective element, see below), Aₘₐₓ is given by:

Aₘₐₓ=(−10/L)*Log₁₀(1−ΔXₘₐₓ) (Eq. 1)

which corresponds to a light-to-heat conversion efficiency 11 of:

η=ΔXₘₐₓ (Eq. 2)

For a double pass embodiment (using a downstream reflective element with reflectivity R), Aₘₐₓ is related to λXₘₐₓ by the following equation which must be solved numerically for Aₘₐₓ:

ΔXₘₐₓ={1+R*10^{-2*A max*L/10}−(1+R)*10^{-A max*L/10}}{1+R*10^{-2*A max*L/10}} (Eq. 3)

This corresponds to a light-to-heat conversion efficiency η of:

η=1−R*10^{-2*A max*L/10}−(1−R)* 10^{-A max*L/10} (Eq. 4)

For instance, if the first region (e.g., doped core) is 1 cm long and one wants the downstream absorbed power to be at most 10% lower than the upstream absorbed power, the maximum attenuation that may be used is Aₘₐₓ=0.45 dB/cm in a single pass embodiment (giving an efficiency of η=10%) and it is Aₘₐₓ=2.0 dB/cm in a double pass embodiment with a 100% reflector (giving an efficiency of η=60%). In some embodiments, it will be desirable to keep ΔX below 50% while maintaining the efficiency above 20%; for a double pass embodiment with a reflector having a reflectivity greater than 90%, this translates into a requirement for the combination A*L to range between about 0.5 dB and about 5.5 dB. In some embodiments, the combination A*L may range between about 0.5 dB and about 5 dB, about 0.5 dB and about 4 dB, about 0.5 dB and about 3 dB, about 0.5 dB and about 2 dB, about 0.5 dB and about 1 dB, about 1 dB and about 5 dB, about 2 dB and about 5 dB, about 3 dB and about 5 dB, or about 4 dB and about 5 dB.

In some embodiments, the dopant is a transition element. In some embodiments, the dopant is Co, Fe, Ni, Cr, Cu, Ti, Mn, V, and/or combinations thereof (e.g., see U.S. Pat. No. 5,572,618 which is incorporated herein by reference in its entirety).

In some embodiments, the dopant concentration affects the absorbance of IR light and attenuation range of the LAF-based device. In some embodiments, the first region (e.g., doped core) absorbs IR light with an attenuation range of about 0.1 dB/cm to about 10 dB/cm. In some embodiments, the first region (e.g., doped core) absorbs IR light with an attenuation range of about 0.1 dB/cm to about 2.5 dB/cm, about 0.5 dB/cm to about 2.5 dB/cm, about 1 dB/cm to about 2.5 dB/cm, or about 1.5 dB/cm to about 2.5 dB/cm.

II. Second Region (e.g., Cladding) that Propagates Visible Light and Enables Evanescent Field Excitation of Its Surface

In some embodiments, the fiber includes a second region (e.g., cladding that surrounds a doped core) that propagates visible light without notable absorption. In some embodiments, a portion of the visible light penetrates the outside of the fiber via an evanescent field and is thus available to excite molecules, when present, on the fiber surface.

Light Sources

In some embodiments, the systems and methods described herein comprise and/or use two light sources. In some embodiments, the systems and methods comprise and/or use three light sources. In some embodiments, a first light source outputs light having wavelengths in the infrared (IR) range. In some embodiments, the IR light has one or more wavelengths from about 750 nm to 50 μm (e.g., from about 880 to 1080 nm, e.g., from about 930 to 1030 nm, e.g., from about 970 to 990 nm, e.g., from about 1300 to 1700 nm, e.g., from about 1552 to 1553 nm). In some embodiments, a second light source outputs light having one or more wavelengths in the visible range. In some embodiments, the transmitted visible light has one or more wavelengths from about 390 to 700 nm (e.g., from about 450 to 500 nm, e.g., about 488 nm). In some embodiments, a third light source outputs light over a range of wavelengths, e.g., a broadband IR light source (e.g., where the range of wavelengths is from about 750 nm to 3 μm). In some embodiments, the third light source is used to interrogate a Fiber Bragg Grating (FBG) inscribed within the first region (e.g., doped core) of the LAF or within a low-loss optical fiber spliced upstream of the LAF. Interrogating the FBG provides an ability to monitor which wavelengths are reflected back by the FBG and which wavelengths are transmitted and this information can be used to measure temperature. In some embodiments, the third light source outputs a single wavelength (e.g., in the range from about 750 nm to 1700 nm, e.g., in the range from about 1525 nm to 1610 nm, e.g., about 1567 nm).

In some embodiments, the first light source includes a variable amplifier (e.g., an erbium-doped fiber amplifier (EDFA)) seeded with a tunable fiber laser adjusted to a first wavelength. In some embodiments, a high power laser diode may be used. In some embodiments, the power output of the first light source can be varied from 0 up to about 20 Watt (e.g., from 0 up to about 2 Watt). In some embodiments, the power output of the second light source is less than about 0.1 Watt (e.g., less than about 0.01 Watt). In some embodiments, the power output of the third light source is in the range of about 0.00001 to about 1 Watt (e.g., about 0.0001 to about 0.1 Watt).

In some embodiments, the second light source includes a laser upstream of an optical shutter and a filter that transmits only the second wavelength. In some embodiments, a fiber pigtailed laser diode is used together with a laser diode driver to enable and disable visible light output.

Evanescent Field Penetration

In some embodiments, visible light is transmitted into the second region (e.g., cladding that surrounds a doped core) of the LAF via a coupling element. The efficiency at which visible light is transmitted into the cladding affects the evanescent field penetration.

In some embodiments, the fluorescence excitation efficiency of light coupled into the second region (e.g., cladding) is dependent on the focusing angle of the light and the spot size of the light at the fiber entrance. In some embodiments, the angle of incidence (e.g., half cone angle) of light at the second wavelength is from about 1 to 45 degrees such as to either maximize and/or minimize the average penetration depth of the evanescent field at the outer surface of the second region (e.g., cladding). In some embodiments, the spot size is from 1 to 1000 μm. One who is skilled in the art would be aware that the optimal angle of incidence and spot size is dependent on the type of fiber and core/cladding dimensions, as well as on the average distance of the fluorophores to be excited from the fiber's surface. In some embodiments, the angle of incidence and spot size are optimized to couple preferentially higher modes or lower modes.

### III. Exemplary Light-Absorbing Optical Fiber (LAF)-Based Device Configurations

The systems and methods can be used in various embodiments and configurations as described in detail herein.

In some embodiments, the LAF is LAF is about 2 mm to 50 mm in length, e.g., about 5 mm to 20 mm in length. In some embodiments, the widest point of a cross section (e.g., diameter) of the LAF is in the range of about 10 μm to 2000 μm, e.g., about 125 μm to 250 μm. In some embodiments, the widest point of a cross section (e.g., diameter) of the first region (e.g., doped core) of the LAF is in the range of about 6μm to 1000 μm, e.g., about 7μm to 50 μm.

Light-Absorbing Optical Fiber (LAF)-Based Device

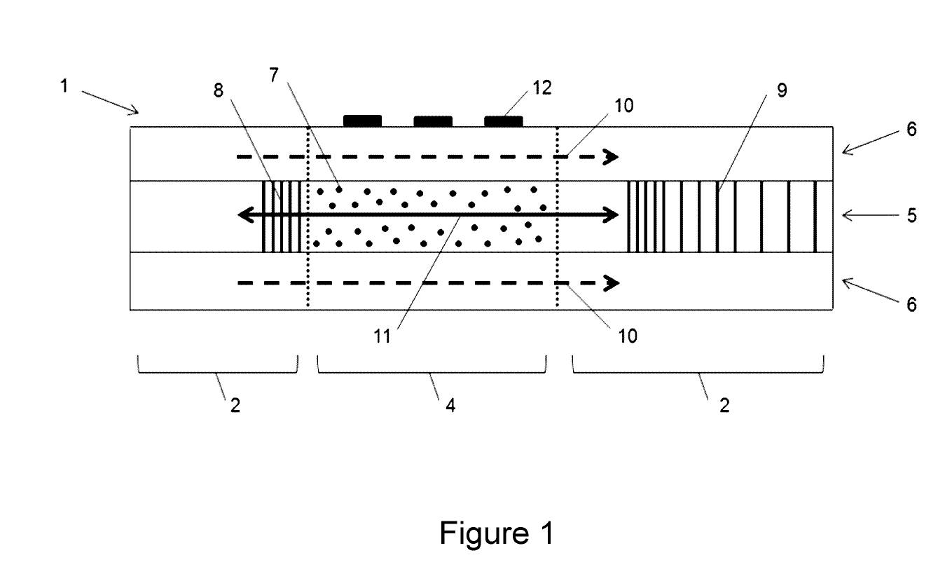

FIG. 1 shows an exemplary light-absorbing optical fiber (LAF)-based device (1). As depicted in FIG. 1, the LAF (4) is flanked by a standard low-loss fiber (2) on each end of the LAF. The LAF comprises a fiber core (5), a fiber cladding (6), a dopant (7), a fiber Bragg grating (FBG) for temperature measurement (8), and a chirped FBG for reflecting light (9). Biomolecules are attached to the cladding surface (12). In this example depicted in FIG. 1, visible light (10) and infrared light (11) are transmitted in the core (5) and cladding (6), respectively.

In some embodiments, a reflective element is placed at the end of the LAF (4). For example, this element may be a gold mirror or a chirped Fiber Bragg Grating (FBG) (9). The element reflects back any IR light that is not absorbed on the first pass through the LAF. There are various advantages to this configuration.

For example, as discussed above, the second pass of IR light may improve the heating efficiency as compared to a single pass.

As another example, the second pass may create a more uniform heating profile. For instance, in the first pass, more light is available to be absorbed at the beginning of the LAF compared to the end, and thus the upstream end heats more than the downstream end, thereby resulting in a thermal gradient that could interfere with efficient amplification (e.g., PCR). However, the second pass results in a thermal gradient in the opposite direction, and this evens out the thermal gradient from the first pass.

As another example, the reflective element may prevent excessive heating at the end of the LAF from leakage of the IR light from the core into the surrounding solution, which could cause another thermal gradient and bubbling.

As another example, in the case of a gold mirror, the reflective element reflects back any visible light in the cladding that was not absorbed on the first pass. This can almost double the potential level of analyte detection (e.g., fluorescent excitation level).

In some embodiments, the reflective element is a chirped FBG selected such that it has a reflection spectrum which transmits IR light at low temperature, not allowing a second pass. As the fiber temperature increases, the chirped FBG spectrum shifts. Eventually, the shifted reflection window of the chirped FBG will match the wavelength of the IR light, and this allows a second pass. Thus, at low temperatures, there will be a larger temperature gradient along the LAF, whereas at high temperatures, there will be more uniform heating. This configuration has various applications. For example, this configuration can be used for multiplex PCR of amplicons that have different annealing temperatures, but similar denaturation temperatures, for example, by placing the amplicons at a spatial location that corresponds to an acceptable annealing temperature. Such a configuration is applicable in the context of surface PCR embodiments, i.e., in cases where the primers attached to the surface of the device are to be extended, not just used for detection. In such an embodiment, primers associated with amplicons that have the highest annealing temperatures are attached upstream of the middle of the LAF section. In such an embodiment, amplicons that have the lowest annealing temperatures are attached downstream of the middle of the LAF section.

In some embodiments, a FBG is inscribed within the LAF and used for temperature monitoring with an additional low-power IR light source and an upstream FBG interrogator (e.g., see Meas. Sci. Technol. 8(4):355-375, 1997 and U.S. Patent Publication No. 2013/0014577 which are both incorporated herein by reference). In some embodiments, precise monitoring of the heating profile across the LAF can be accomplished by inscribing multiple adjacent FBGs with distinct periods within the LAF, and using the multiple adjacent FBGs to provide multiple local temperature measurements. There are some potential disadvantages of inscribing the FBG within the LAF in the context of the presented systems and methods in that temperature readability can be lowered because of the reduction in Bragg peak amplitude resulting from light absorption in a LAF core and from the lower efficiency of grating inscription in a LAF compared to standard fiber. The temperature readability is lowered if there is a reflective element at the end of the LAF because of an increase in the entire baseline around the Bragg peak, thereby reducing visibility. Moreover, FBG inscription often results in UV-induced photoluminescence, which also provides an undesired background during fluorescence measurements of biomolecules (see J. Appl. Physics. 116: 064906, 2014).

In some embodiments, a FBG can therefore be inscribed in an upstream standard low-loss fiber just before the LAF. In contrast to inscribing a FBG within the LAF, using an FBG inscribed just before the LAF allows the use of low-cost draw-tower gratings inscribed within standard low-loss fiber prior to the device assembly, thereby reducing manufacturing costs significantly.

Free Space Coupling

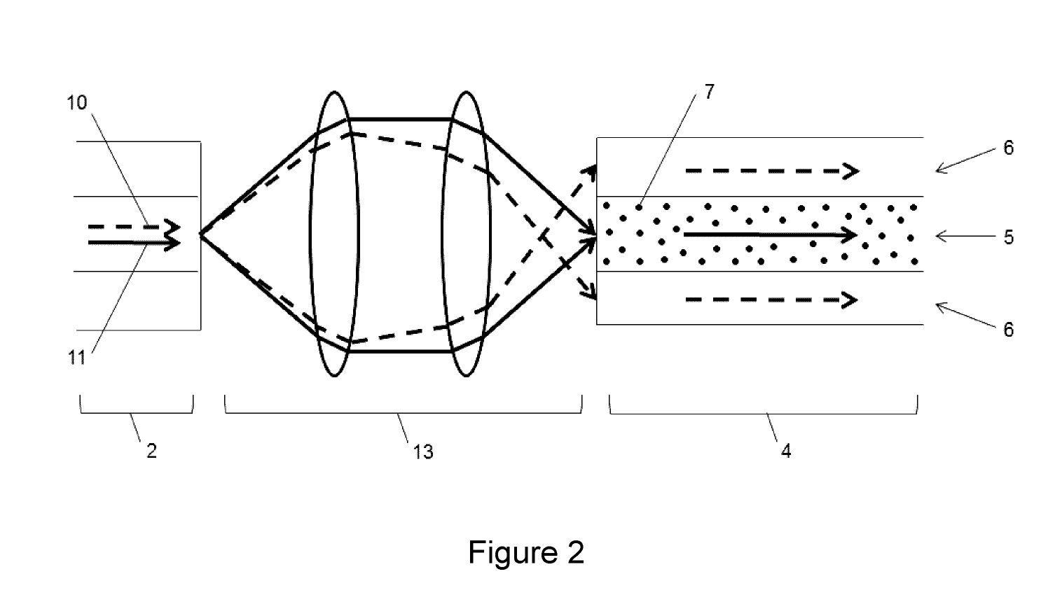

In some embodiments, the visible and IR light sources are initially multiplexed in the core of a single fiber input. In some embodiments, a free space coupler (FIG. 2) (13) is used to couple the multiplex light from the single fiber input into the LAF. For example, the IR light (11) goes into the core (5) of the LAF and the visible light (10) goes into the cladding (6) due to the lenses' refractive index dependence on wavelength. One potential advantage of using a free space coupler is it is a low-cost method of coupling multiple light sources into the core and cladding. Another potential advantage is that air flow across the coupler may be used to blow away dust from the ends of the single fiber input and the LAF. In contrast, in-fiber coupling options involving physical contact of two distinct fiber ends can have issues with dust burning onto the ends of the fibers.

In some embodiments, the free space coupler design (e.g., lenses geometry and/or glass type) is optimized with respect to the evanescent field fluorescence excitation for enabling optimal PCR monitoring. In some embodiments, the average penetration depth of the evanescent field is minimized and/or maximized depending on the detection strategy used (e.g., the average distance of the fluorescent molecules of interest with respect to the fiber surface can vary from a few nanometers to beyond hundreds of nanometers). In some embodiments, the average penetration depth of the evanescent field is minimized and/or maximized depending on the presence or absence of nanoparticle-based fluorescence enhancement. Since different transverse cladding modes have different penetration depths for a given wavelength, the coupling efficiency from the free space coupler to the various cladding modes can impact the fluorescence excitation efficiency. By adjusting the chromatic aberration of the free space coupler, the coupling efficiency to the various cladding modes can be adjusted for the visible light while ensuring optimal core coupling for the IR light source(s). Further, these considerations can remain relevant in the presence of a double-cladding low-loss fiber segment spliced upstream of the LAF (see below) and in the context of completely in-fiber coupling (see below). In the latter case, the design of the wavelength-dependent core-to-cladding coupling element can be optimized to favor certain cladding modes for the visible light.

Standard Fiber Spliced Upstream of the LAF and Example of Capillary Reaction Vessel

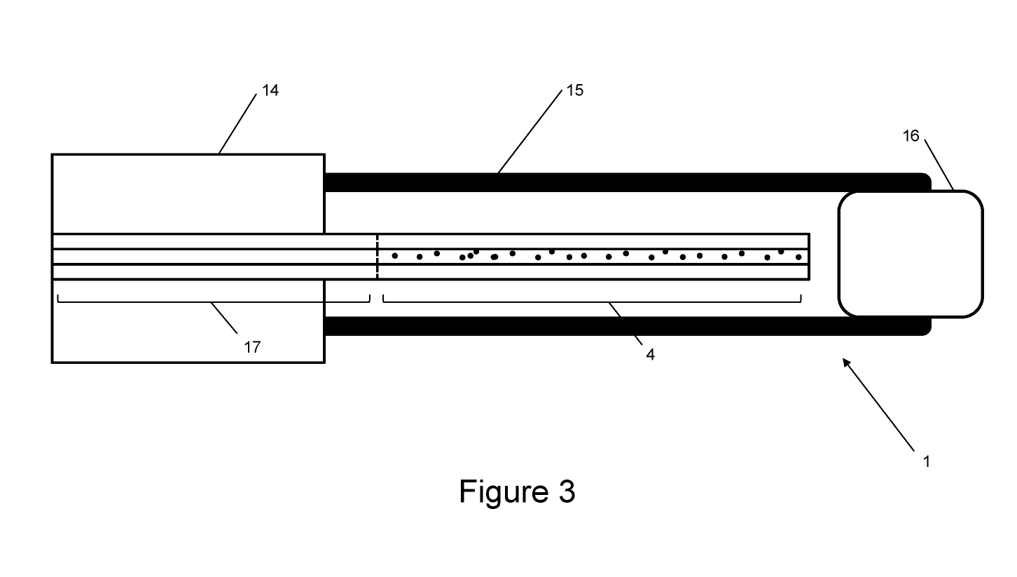

In some embodiments, a standard or double-cladding low-loss fiber is spliced upstream of the LAF. The low-loss fiber then transmits visible light through its inner or outer cladding, and IR light through its core, into the respective cladding and core of the LAF. An advantage of this configuration is that a ferrule or other support structure can be used to hold the standard fiber in place (FIG. 3). In contrast, if a ferrule were to hold an LAF in place, heat would be generated and conducted from the LAF to the ferrule, and this would waste IR power. Further, the conducted heat could potentially weaken the cartridge assembly by heating and softening the glue binding the ferrule and LAF. Using a double-cladding fiber in which the visible light propagates through an inner cladding also has the added advantage compared to a standard fiber that the ferrule causes no attenuation and no undesired scattering into the reaction vessel.

FIG. 3 shows an exemplary configuration of a fiber device (1). In this configuration, a standard fiber (17) is spliced upstream of the LAF (4). The fiber device (1) is held by a ferrule (14) at the standard fiber (17) portion of the device. The LAF (4) portion of the device is contained within a capillary reaction vessel (15). The capillary vessel (15) is closed via a capillary cap (16).

Multimode Interference Section for Completely In-Fiber Coupling

In an example configuration where visible and IR light sources are initially multiplexed in the core of a single fiber input and completely in-fiber coupling is desired, various schemes can be used to selectively couple the majority of the visible light into the cladding and the majority of the IR light into the core of the device. In some embodiments, a multimode interference (MMI) section can be spliced upstream of the LAF fiber (see Optical Engineering. 47.11: 112001-112001, 2008). Such an MMI section can simultaneously act as the low-loss fiber section allowing a ferrule or other support structure to hold and center the device in a capillary reaction vessel.

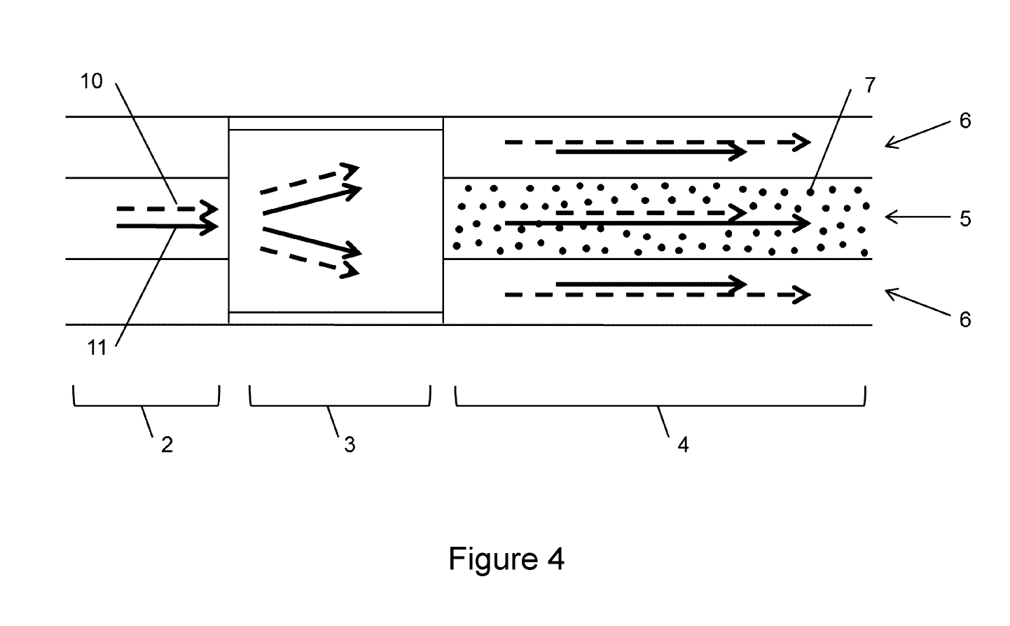

FIG. 4 shows an example configuration where a MMI section is used for completely in-fiber coupling. In this example configuration, a single mode fiber (SMF) (2) is spliced upstream of a multimode fiber (MMF) (3). The MMF (3) is spliced upstream of the LAF (4). As IR light (11) and visible light (10) is transmitted through the SMF (2) and (MMF) (3), the IR light (11) and visible light (10) is multiplexed into the fiber core (5) containing a dopant (7) and fiber cladding (6).

In some embodiments, the core of the low-loss multimode optical fiber is larger than the core of the LAF.

Nanoparticle Coating

In some embodiments, nanoparticles can be coated on the surface of the LAF section, prior to coating the biomolecules, in order to enhance the evanescent field fluorescence excitation. Examples of nanoparticle coatings on a glass substrate (e.g., glass slide or glass bead) for fluorescence enhancement purposes can be found, for example, in Abel, Biebele, et al. “Metal-Enhanced Fluorescence from Silver Nanowires with High Aspect Ratio on Glass Slides for Biosensing Applications.” The Journal of Physical Chemistry C 119.1 (2014): 675-684, or Goldys, Ewa M., and Fang Xie. “Metallic nanomaterials for sensitivity enhancement of fluorescence detection.” Sensors 8.2 (2008): 886-896, which are both incorporated herein by reference. In the context of the present invention, the use of nanoparticle coating-based fluorescence enhancement can lead to lower power requirements for the visible light source and/or to a greater sensitivity.

### IV. Capture Probes Immobilized on an Outside Surface of the Second Region (e.g., Cladding)

Systems and methods of the present invention are generally applicable to any capture probes.

In some embodiments, capture probes (e.g., biomolecules that are immobilized on the surface of the second region, e.g., cladding) bind to an analyte in a sample (e.g., a biological sample). In some embodiments, the analytes are labeled with a fluorophore or other detectable marker. In some embodiments, the bound analytes are detected using secondary detection probes (e.g., fluorophores that bind with double-stranded nucleic acids such as SYBR Green or secondary biomolecules, e.g., labeled oligonucleotides or labeled antibodies that are labeled, e.g., with a fluorophore or other detectable marker). Such detection of analytes is well-known in the art. By way of example, in some embodiments, nucleic acid capture probes are spotted at different locations on an outside surface of the second region (e.g., cladding) of the LAF, which is exposed to a sample containing an intercalating dye such as SYBR Green. When the nucleic acid capture probes hybridize with a complementary polynucleotide (e.g., a nucleic acid analyte), the intercalating dye binds to the resulting double-stranded construct. This construct then fluoresces when excited by the evanescent field which is generated at the surface of the second region (e.g., cladding) when visible light is transmitted through the second region. In some embodiments, the penetration depth of the evanescent field is approximately 1 micrometer, and there is therefore a significant signal-to-background contrast because the rest of the intercalating dye in the surrounding sample solution is not excited—only the dye at the surface of the second region (e.g., cladding) is excited.

Biomolecules

As will be understood by a person of ordinary skill in the art, in some embodiments, capture probes may include polynucleotides (e.g., RNA, DNA, PNA, oligonucleotide that are at least partially complementary to the analyte), polypeptides (e.g., antibodies or receptors that bind the analyte), polysaccharides (e.g., that are bound by the analyte), or a combination thereof.

Fluorescent Reporter

In some embodiments, methods detect analytes via a fluorescent reporter that is attached to the captured analyte (either directly or via a secondary detection probe). In some embodiments, the fluorescent reporter is a near infrared or far infrared dye. In some embodiments, the fluorescent reporter is selected from the group consisting of a fluorophore, fluorochrome, dye, pigment, fluorescent transition metal, molecular beacon, and fluorescent protein. In some embodiments, the fluorescent reporter is selected from the group consisting of Cy5, Cy5.5, Cy2, FITC, TRITC, Cy7, FAM, Cy3, Cy3.5, Texas Red, ROX, HEX, JA133, AlexaFluor 488, AlexaFluor 546, AlexaFluor 633, AlexaFluor 555, AlexaFluor 647, DAPI, TMR, R6G, GFP, enhanced GFP, CFP, ECFP, YFP, Citrine, Venus, YPet, CyPet, AMCA, Spectrum Green, Spectrum Orange, Spectrum Aqua, Lissamine and Europium. In some embodiments, detecting is performed in normal lighting settings. In some embodiments, detecting is performed with some to zero levels of ambient lighting settings.

The methods herein can be used with a number of different fluorescent reporters (or, as in embodiments using a tandem bioluminescent reporter/fluorescent probe, the fluorescent species thereof), for example, (1) detection probes that become activated after binding or interacting with an analyte (Weissleder et al., Nature Biotech., 17:375-378, 1999; Bremer et al., Nature Med., 7:743-748, 2001; Campo et al., Photochem. Photobiol. 83:958-965, 2007); (2) wavelength shifting beacons (Tyagi et al., Nat. Biotechnol., 18:1191-1196, 2000); (3) multicolor, e.g., fluorescent detection probes (Tyagi et al., Nat. Biotechnol., 16:49-53, 1998); (4) detection probes that have high binding affinity to the analyte (Achilefu et al., Invest. Radiol., 35:479-485, 2000; Becker et al., Nature Biotech. 19:327-331, 2001; Bujai et al., J. Biomed. Opt. 6:122-133, 2001; Ballou et al. Biotechnol. Prog. 13:649-658, 1997; and Neri et al., Nature Biotech 15:1271-1275, 1997); (5) quantum dot or nanoparticle-based detection probes, including multivalent detection probes, and fluorescent quantum dots such as amine T2 MP EviTags® (Evident Technologies) or Qdot® Nanocrystals (Invitrogen™); and/or (6) non-specific detection probes, e.g., indocyanine green, AngioSense® (VisEn Medical). The relevant text of the above-referenced documents are incorporated by reference herein. Another group of suitable detection probes are lanthanide metal-ligand probes. In general, fluorescent quantum dots used in the practice of the elements of this invention are nanocrystals containing several atoms of a semiconductor material (including but not limited to those containing cadmium and selenium, sulfide, or tellurium; zinc sulfide, indium-antimony, lead selenide, gallium arsenide, and silica or ormosil), which have been coated with zinc sulfide to improve the properties of the fluorescent agents.

In particular, fluorescent probe species are a preferred type of probe. A fluorescent probe species is a fluorescent probe that is targeted to an analyte, molecular structure or biomolecule, such as a cell-surface receptor or antigen, an enzyme within a cell, or a specific nucleic acid, e.g., DNA, to which the probe hybridizes. Biomolecules that can be targeted by fluorescent imaging probes include, for example, antibodies, proteins, glycoproteins, cell receptors, neurotransmitters, integrins, growth factors, cytokines, lymphokines, lectins, selectins, toxins, carbohydrates, internalizing receptors, enzyme, proteases, viruses, microorganisms, and bacteria.

In some embodiments, probe species have excitation and emission wavelengths in the red and near infrared spectrum, e.g., in the range 550-1300 or 400-1300 nm or from about 440 to about 1100 nm, from about 550 to about 800 nm, or from about 600 to about 900 nm. Probe species with excitation and emission wavelengths in other spectrums, such as the visible and ultraviolet light spectrum, can also be employed in the methods of the embodiments of the present invention. In particular, fluorophores such as certain carbocyanine or polymethine fluorescent fluorochromes or dyes can be used to construct optical imaging agents, e.g. U.S. Pat. Nos. 6,747,159; 6,448,008; 6,136,612; 4,981,977; 5,268,486; 5,569,587; 5,569,766; 5,486,616; 5,627,027; 5,808,044; 5,877,310; 6,002,003; 6,004,536; 6,008,373; 6,043,025; 6,127,134; 6,130,094; 6,133,445; 7,445,767; 6,534,041; 7,547,721; 7,488,468; 7,473,415; also WO 96/17628, EP 0 796 111 B1, EP 1 181 940 B1, EP 0 988 060 B1, WO 98/47538, WO 00/16810, EP 1 113 822 B1, WO 01/43781, EP 1 237 583 A1, WO 03/074091, EP 1 480 683 B1, WO 06/072580, EP 1 833 513 A1, EP 1 679 082 A1, WO 97/40104, WO 99/51702, WO 01/21624, and EP 1 065 250 A1; and Tetrahedron Letters 41:9185-9188, 2000.

Exemplary fluorochromes for detection probes include, for example, the following: Cy5.5, CyS, Cy7.5 and Cy7 (GE® Healthcare); AlexaFluor660, AlexaFluor680, AlexaFluor790, and AlexaFluor750 (Invitrogen); VivoTag™680, VivoTag™-S680, VivoTag™-S750 (VISEN Medical); Dy677, Dy682, Dy752 and Dy780 (Dyomics®); DyLight® 547, and/or DyLight® 647 (Pierce); HiLyte Fluor™ 647, HiLyte Fluor™ 680, and HiLyte Fluor™ 750 (AnaSpec®) IRDye® 800CW, IRDye® 800RS, and IRDye® 700DX (Li-Cor®); ADS780WS, ADS830WS, and ADS832WS (American Dye Source); XenoLight CF™ 680, XenoLight CF™ 750, XenoLight CF™ 770, and XenoLight DiR (Caliper® Life Sciences); and Kodak® X-SIGHT® 650, Kodak® X-SIGHT 691, Kodak® X-SIGHT 751 (Carestream® Health).

Amplification Conditions

In some embodiments, systems and methods of the present disclosure are used to detect a nucleic acid analyte via polymerase chain reactions (PCR) (e.g., qPCR). Conditions that allow amplification of a nucleic acid analyte comprise incubating a reaction mixture through one or more cycles of different temperatures (“thermocycling”). Exemplary amplification techniques in which the reaction is temperature dependent include, but are not limited to, PCR, qPCR, and asymmetric PCR.

In some embodiments, conditions that allow amplification of a nucleic acid analyte comprise incubating a reaction mixture through one or more cycles of maintained temperature (“isothermal”). In some embodiments, amplification techniques in which the reaction is not temperature dependent include, but are not limited to, isothermal PCR and nucleic acid sequence based amplification (NASBA), and strand displacement amplification (SDA).

In some embodiments, detection of nucleic acid analytes with the systems and methods described herein comprises amplifying a nucleic acid analyte in the sample and using immobilized capture probes on an area of the surface of the second region (e.g., cladding) of the LAF to capture the amplified nucleic acid analyte. Such embodiments generally include the use of asymmetric PCR in solution to overproduce one of the two strands, for example, the strand that is complementary to the surface-immobilized primers. In some cases, a fraction of the overproduced strands are free to hybridize to the surface-immobilized oligonucleotides. In some embodiments, amplification and detection of nucleic acid analytes with the systems and methods described herein comprises amplifying a nucleic acid analyte on the surface of the second region (e.g., cladding) of the LAF (i.e., using amplification primers that are immobilized on the fiber itself in addition to the amplification primers that are in solution).

In some embodiments, such conditions are similar to or the same as conditions suitable for traditional PCR. For example, a reaction mixture can undergo one or more thermocycles, each thermocycle comprising incubating the reaction mixture at 1) a temperature suitable for denaturation of double-stranded polynucleotide complexes for a period of time (the “denaturation phase”), 2) then at a temperature suitable for annealing of two strands of polynucleotides and/or oligonucleotides (e.g., annealing of oligonucleotides to a polynucleotide) for a period of time (the “annealing phase”), and 3) then at a temperature suitable for extension of an oligonucleotide primer by one or more nucleotides for a period of time (the “extension phase”), wherein the periods of time during the denaturation phase, the annealing phase, and the extension phase may be different or the same.

In some embodiments, the reaction mixture is incubated for an initial period of time at the temperature suitable for denaturation before the set of thermocycles.

In some embodiments, the reaction mixture is incubated for a final period of time at the temperature suitable for extension after the set of thermocycles.

The temperatures, periods of time for each temperature, and the total number of thermocycles may vary depending on the embodiment and/or may be influenced by factors such as the length of the template polynucleotide, complexity of the template polynucleotide sequence, type of DNA polymerase used, etc. For example, a longer extension phase (e.g., longer than the denaturation phase and/or the annealing phase) might be used when trying to amplify a larger nucleotide sequence.

In general, one of ordinary skill in the art would be able to adjust the conditions for amplification accordingly.

In some embodiments, the temperature used during the denaturation phase (the “denaturation temperature”) is within a range of from about −20 to +2 degrees Celsius of 97° C., within a range of from about −10 to +1 degrees Celsius of 97° C., within a range of from about −5 to about +0.5 degrees Celsius of 97° C. In some embodiments, the denaturation temperature is approximately 95° C.

In some embodiments, the temperature used during the annealing phase (the “annealing temperature”) is within fifteen degrees Celsius of 55° C., within ten degrees Celsius of 55° C., within five degrees Celsius of 55° C., within four degrees Celsius of 55° C., within three degrees Celsius of 55° C., within two degrees Celsius of 55° C., or within one degree Celsius of 55° C. In some embodiments, the annealing temperature is approximately 55° C.

In some embodiments, the temperature used during the extension phase (the “extension temperature”) is within fifteen degrees Celsius of 65° C., within ten degrees Celsius of 65° C., within five degrees Celsius of 72° C., within four degrees Celsius of 72° C., within three degrees Celsius of 72° C., within two degrees Celsius of 72° C., or within one degree Celsius of 72° C. In some embodiments, the extension temperature is approximately 72° C. In some embodiments, the extension temperature is as defined above for the annealing temperature. In some embodiments, the extension temperature and the annealing temperature are the same, e.g., within five degrees Celsius of 55° C., within three degrees Celsius of 55° C., or within one degree Celsius of 55° C. In some embodiments, the extension temperature and annealing temperature are approximately 55° C.

In some embodiments, the temperature used during a combined annealing/extension phase (the “annealing/extension temperature”) is within twenty degrees Celsius of 60° C., within fifteen degrees Celsius of 60° C., within ten degrees Celsius of 60° C., within five degrees Celsius of 60° C., within four degrees Celsius of 60° C., within three degrees Celsius of 60° C., within two degrees Celsius of 60° C., or within one degree Celsius of 60° C. In some embodiments, the annealing/extension temperature is approximately 55° C.

In some embodiments, the length of the denaturation phase (the “denaturation period”) is greater than about 0.1 second, greater than about 1 second greater, greater than about 5 seconds, greater than about 15 seconds, greater than about 20 seconds, or greater than about 25 seconds. In some embodiments, the denaturation period is less than about 50 seconds, less than about 45 seconds, less than about 40 seconds, or less than about 30 seconds. In some embodiments, the denaturation period is about 30 seconds.

In some embodiments, the length of the annealing phase (the “annealing period”) is greater than about 1 second, greater than about 15 seconds, greater than about 20 seconds, or greater than about 25 seconds. In some embodiments, the annealing period is less than about 50 seconds, less than about 45 seconds, less than about 40 seconds, or less than about 30 seconds. In some embodiments, the annealing period is about 30 seconds.

The length of the extension phase (the “extension period”) can be varied, e.g., depending on the length of the nucleotide sequence that is being amplified. Generally, a longer extension period may be suitable for longer nucleotide sequences. Moreover, the extension phase for the last cycle can be longer than the extension phase for the rest of the cycles.

In some embodiments, the length of the annealing/extension phase (the “annealing/extension period”) is greater than about 0.1 second, greater than about 1 second, greater than about 5 seconds, greater than about 15 seconds, greater than about 20 seconds, or greater than about 25 seconds. In some embodiments, the annealing/extension period is less than about 50 seconds, less than about 45 seconds, less than about 40 seconds, or less than about 30 seconds. In some embodiments, the annealing period is about 30 seconds.

In some embodiments, the extension period for the last cycle is greater than about 1 second, greater than about 30 seconds, greater than about 60 seconds, greater than about 90 seconds, greater than about 2 minutes, greater than about 2.5 minutes, greater than about 3 minutes, greater than about 3.5 minutes, greater than about 4 minutes, greater than about 4.5 minutes, greater than about 5 minutes, greater than about 5.5 minutes, greater than about 6 minutes, greater than about 6.5 minutes, greater than about 7 minutes, greater than about 7.5 minutes, greater than about 8 minutes, greater than about 8.5 minutes, or greater than about 9 minutes. In some embodiments, the extension period for all but the last cycles is greater than about 1 second, greater than about 15 seconds, greater than about 30 seconds, or greater than about 60 seconds.

In some embodiments, the extension period for the last cycle is less than about 20 minutes, less than about 19 minutes, less than about 18 minutes, less than about 17 minutes, less than about 16 minutes, less than about 15 minutes, less than about 14 minutes, less than about 13 minutes, less than about 12 minutes, or less than about 11 minutes. In some embodiments, the extension period for the last cycle is about 10 minutes. In some embodiments, the extension period for all but the last cycle is less than 1 minute, or less than 10 seconds.

Multiplex Capacity

In some embodiments, the systems and methods described herein provide the ability to detect multiple analytes in a single test for multiplex detection capability.

In some embodiments, at least 2, at least 5, at least 10, at least 50, at least 100, at least 1000, at least 100000, or at least 1000000000 capture probes of the same type or of different types (e.g., 2, 3, 4, or 5 types) are immobilized on an area of the surface of the second region (e.g., cladding) to detect an analyte or multiple analytes in solution. Such groupings of capture probes of the same type or of different type that are immobilized on a specific area of the surface of the LAF constitute one set (e.g., one spot). In some embodiments, multiple (e.g., more than 1, more than 5, more than 10, or more than 100) spots of immobilized capture probes exist, each associated with one or multiple distinct analytes in solution, and are organized in an array format (e.g., adjacent spots spacially separated) on an outside surface of the second region (e.g., cladding) of the LAF. In some embodiments, detection of a plurality of distinct analytes is performed simultaneously. In some embodiments, detection of a plurality of distinct analytes is performed in real-time.

In some embodiments, when multiple distinct sets of capture probes are immobilized on an area of the surface of the second region (e.g., cladding), multiple corresponding sets of detection probes (e.g., fluorescently labeled single-stranded oligonucleotides) may be used where each set of detection probes includes a detectable marker (e.g., different fluorescent reporter). In embodiments in which each set (e.g., each spot) is composed of a single type of capture probe, the signal produced by the markers associated with the different capture probes does not need to be distinguishable spectrally from one another as the signals are spatially separated. In such embodiments, alternatively to the use of fluorescently labeled single-stranded oligonucleotides as detection probes, an intercalating dye can be used as multiplexed detection of different analytes is enabled by the spatial separation of the corresponding sets of capture probes.

In embodiments in which each set comprises more than a single type of capture probe, the signals produced by the markers associated with the different capture probes included in a single set must be distinguishable spectrally (e.g., fluorescent signals at different wavelengths).

### V. Illustrative Network Environment

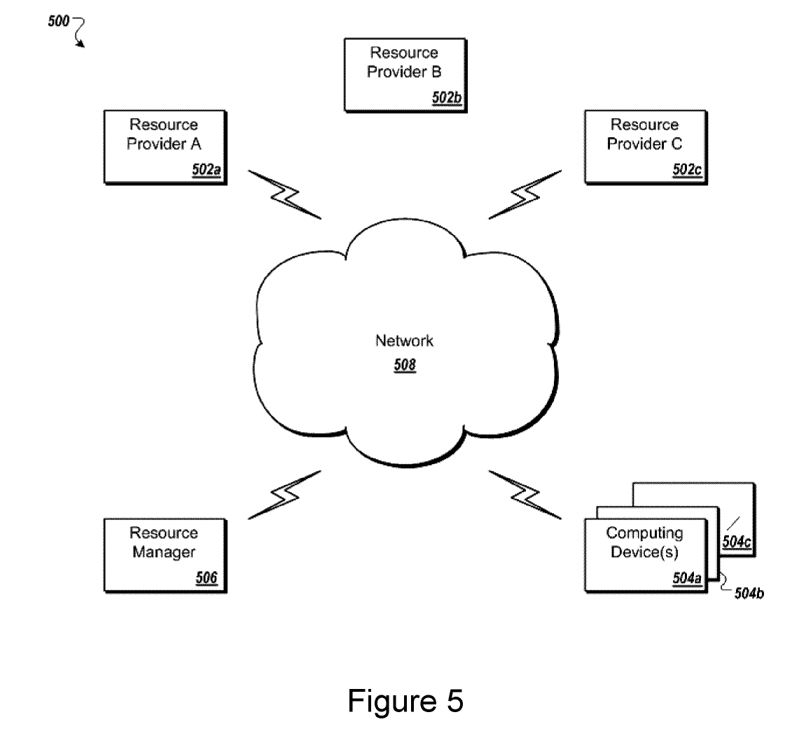

FIG. 5 shows an illustrative network environment 500 for use in analysis of data (e.g., spectrometry signals, e.g., fluorescent signals, e.g., temperature profiles) acquired by optical fiber-based systems and methods described herein. In brief overview, referring now to FIG. 5, a block diagram of an exemplary cloud computing environment 500 is shown and described. The cloud computing environment 500 may include one or more resource providers 502a, 502b, 502c (collectively, 502). Each resource provider 502 may include computing resources. In some implementations, computing resources may include any hardware and/or software used to process data (e.g., data acquired by an optical fiber-based device (e.g., a portable optical fiber-based device)). For example, computing resources may include hardware and/or software capable of executing algorithms, computer programs, and/or computer applications (e.g., related to analyzing data (e.g., spectrometry signals, e.g., fluorescent signals, e.g., temperature profiles) acquired by optical fiber-based devices described herein). In some implementations, exemplary computing resources may include application servers and/or databases with storage and retrieval capabilities (e.g., for storing data acquired by the optical fiber-based devices described herein). Each resource provider 502 may be connected to any other resource provider 502 in the cloud computing environment 500. In some implementations, the resource providers 502 may be connected over a computer network 508. Each resource provider 502 may be connected to one or more computing device 504a, 504b, 504c (collectively, 504), over the computer network 508.

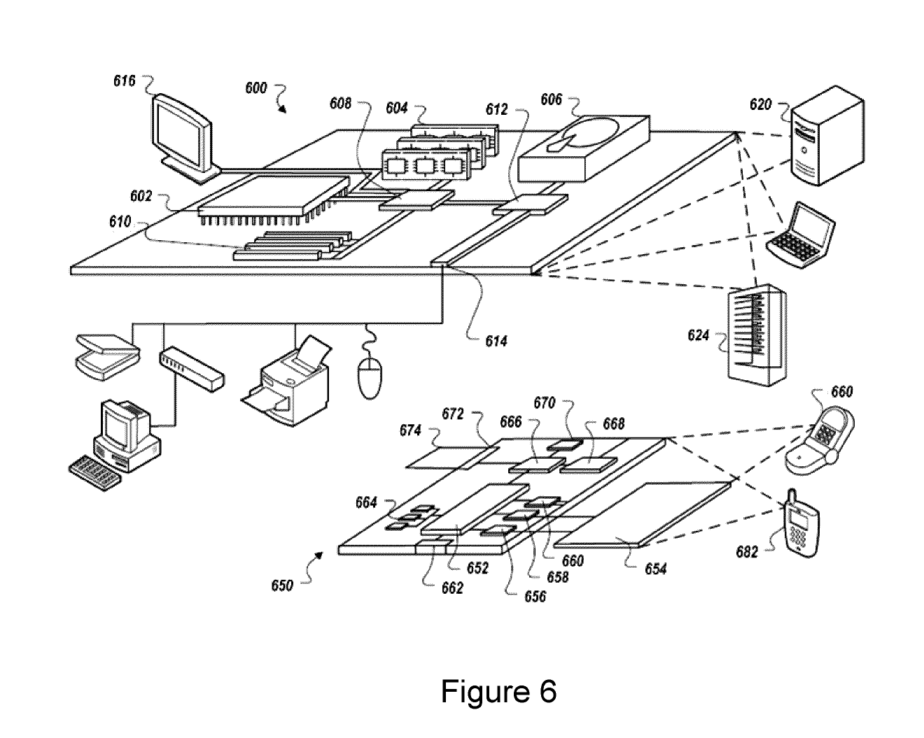

FIG. 6 shows an example of a computing device 600 and a mobile computing device 650 that can be used in analysis of data (e.g., spectrometry signals, e.g., fluorescent signals, e.g., temperature profiles) acquired by optical fiber-based systems and methods, as described herein. The computing device 600 is intended to represent various forms of digital computers, such as laptops, desktops, workstations, servers, blade servers, mainframes, portable devices, and other appropriate computers. The mobile computing device 650 is intended to represent various forms of mobile devices, such as personal digital assistants, cellular telephones, smart-phones, and other similar computing devices. For example, data acquired by an exemplary optical fiber-based device can be accessed and/or presented by the computer device 600 and/or mobile computing device 650. The components shown here, their connections and relationships, and their functions, are meant to be examples only, and are not meant to be limiting.

The computing device 600 includes a processor 602, a memory 604, a storage device 606, a high-speed interface 608 connecting to the memory 604 and multiple high-speed expansion ports 610, and a low-speed interface 612 connecting to a low-speed expansion port 614 and the storage device 606. Each of the processor 602, the memory 604, the storage device 606, the high-speed interface 608, the high-speed expansion ports 610, and the low-speed interface 612, are interconnected using various busses, and may be mounted on a common motherboard or in other manners as appropriate. The processor 602 can process instructions (e.g., instructions (e.g., power output) triggered by measured temperature profiles by the optical fiber-based devices provided herein) for execution within the computing device 600, including instructions stored in the memory 604 or on the storage device 606 to display graphical information for a GUI on an external input/output device, such as a display 616 coupled to the high-speed interface 608. In other implementations, multiple processors and/or multiple buses may be used, as appropriate, along with multiple memories and types of memory. Also, multiple computing devices may be connected, with each device providing portions of the necessary operations (e.g., as a server bank, a group of blade servers, or a multi-processor system).

The memory 604 stores information (e.g., data, e.g., fluorescent signals, e.g., temperature profiles collected by a sensor of the optical fiber-based device) within the computing device 600. In some implementations, the memory 604 is a volatile memory unit or units. In some implementations, the memory 604 is a non-volatile memory unit or units. The memory 604 may also be another form of computer-readable medium, such as a magnetic or optical disk.

The storage device 606 is capable of providing mass storage for the computing device 600. In some implementations, the storage device 606 may be or contain a computer-readable medium, such as a a hard disk device, an optical disk device, or a tape device, a flash memory or other similar solid state memory device, or an array of devices, including devices in a storage area network or other configurations. Instructions can be stored in an information carrier. The instructions, when executed by one or more processing devices (for example, processor 602), perform one or more methods, such as those described above. The instructions can also be stored by one or more storage devices such as computer- or machine-readable mediums (for example, the memory 604, the storage device 606, or memory on the processor 602).

The high-speed interface 608 manages bandwidth-intensive operations for the computing device 600, while the low-speed interface 612 manages lower bandwidth-intensive operations. Such allocation of functions is an example only. In some implementations, the high-speed interface 608 is coupled to the memory 604, the display 616 (e.g., through a graphics processor or accelerator), and to the high-speed expansion ports 610, which may accept various expansion cards (not shown). In the implementation, the low-speed interface 612 is coupled to the storage device 606 and the low-speed expansion port 614. The low-speed expansion port 614, which may include various communication ports (e.g., USB, Bluetooth®, Ethernet, wireless Ethernet) may be coupled to one or more input/output devices, such as a keyboard, a pointing device, a scanner, or a networking device such as a switch or router, e.g., through a network adapter.

The computing device 600 may be implemented in a number of different forms, as shown in the figure. For example, it may be implemented as a standard server 620, or multiple times in a group of such servers. In addition, it may be implemented in a personal computer such as a laptop computer 622. In addition, it may be implemented in a portable device containing an optical fiber-based device as described herein. It may also be implemented as part of a rack server system 624. Alternatively, components from the computing device 600 may be combined with other components in a mobile device (not shown), such as a mobile computing device 650. Each of such devices may contain one or more of the computing device 600 and the mobile computing device 650, and an entire system may be made up of multiple computing devices communicating with each other.

The mobile computing device 650 includes a processor 652, a memory 664, an input/output device such as a display 654, a communication interface 666, and a transceiver 668, among other components. The mobile computing device 650 may also be provided with a storage device, such as a micro-drive or other device, to provide additional storage. Each of the processor 652, the memory 664, the display 654, the communication interface 666, and the transceiver 668, are interconnected using various buses, and several of the components may be mounted on a common motherboard or in other manners as appropriate.

The processor 652 can execute instructions within the mobile computing device 650, including instructions stored in the memory 664. The processor 652 may be implemented as a chipset of chips that include separate and multiple analog and digital processors. The processor 652 may provide, for example, for coordination of the other components of the mobile computing device 650, such as control of user interfaces, applications run by the mobile computing device 650, and wireless communication by the mobile computing device 650.

The processor 652 may communicate with a user through a control interface 658 and a display interface 656 coupled to the display 654. The display 654 may be, for example, a TFT (Thin-Film-Transistor Liquid Crystal Display) display or an OLED (Organic Light Emitting Diode) display, or other appropriate display technology. The display interface 656 may comprise appropriate circuitry for driving the display 654 to present graphical and other information to a user. The control interface 658 may receive commands from a user and convert them for submission to the processor 652. In addition, an external interface 662 may provide communication with the processor 652, so as to enable near area communication of the mobile computing device 650 with other devices. The external interface 662 may provide, for example, for wired communication in some implementations, or for wireless communication in other implementations, and multiple interfaces may also be used.

The memory 664 stores information within the mobile computing device 650. The memory 664 can be implemented as one or more of a computer-readable medium or media, a volatile memory unit or units, or a non-volatile memory unit or units. An expansion memory 674 may also be provided and connected to the mobile computing device 650 through an expansion interface 672, which may include, for example, a SIMM (Single In Line Memory Module) card interface. The expansion memory 674 may provide extra storage space for the mobile computing device 650, or may also store applications or other information for the mobile computing device 650. Specifically, the expansion memory 674 may include instructions to carry out or supplement the processes described above, and may include secure information also. Thus, for example, the expansion memory 674 may be provided as a security module for the mobile computing device 650, and may be programmed with instructions that permit secure use of the mobile computing device 650. In addition, secure applications may be provided via the SIMM cards, along with additional information, such as placing identifying information on the SIMM card in a non-hackable manner.

The memory may include, for example, flash memory and/or NVRAM memory (non-volatile random access memory), as discussed below. In some implementations, instructions are stored in an information carrier and, when executed by one or more processing devices (for example, processor 652), perform one or more methods, such as those described above. The instructions can also be stored by one or more storage devices, such as one or more computer- or machine-readable mediums (for example, the memory 664, the expansion memory 674, or memory on the processor 652). In some implementations, the instructions can be received in a propagated signal, for example, over the transceiver 668 or the external interface 662.

The mobile computing device 650 may communicate wirelessly through the communication interface 666, which may include digital signal processing circuitry where necessary. The communication interface 666 may provide for communications under various modes or protocols, such as GSM voice calls (Global System for Mobile communications), SMS (Short Message Service), EMS (Enhanced Messaging Service), or MMS messaging (Multimedia Messaging Service), CDMA (code division multiple access), TDMA (time division multiple access), PDC (Personal Digital Cellular), WCDMA (Wideband Code Division Multiple Access), CDMA2000, or GPRS (General Packet Radio Service), among others. Such communication may occur, for example, through the transceiver 668 using a radio-frequency. In addition, short-range communication may occur, such as using a Bluetooth®, Wi-Fi™, or other such transceiver (not shown). In addition, a GPS (Global Positioning System) receiver module 670 may provide additional navigation- and location-related wireless data to the mobile computing device 650, which may be used as appropriate by applications running on the mobile computing device 650.

The mobile computing device 650 may also communicate audibly using an audio codec 660, which may receive spoken information from a user and convert it to usable digital information. The audio codec 660 may likewise generate audible sound for a user, such as through a speaker, e.g., in a handset of the mobile computing device 650. Such sound may include sound from voice telephone calls, may include recorded sound (e.g., voice messages, music files, etc.) and may also include sound generated by applications operating on the mobile computing device 650.

The mobile computing device 650 may be implemented in a number of different forms, as shown in the figure. For example, it may be implemented as a cellular telephone 680. It may also be implemented as part of a smart-phone 682, personal digital assistant, or other similar mobile device.

Various implementations of the systems and methods described here can be realized in digital electronic circuitry, integrated circuitry, specially designed ASICs (application specific integrated circuits), computer hardware, firmware, software, and/or combinations thereof These various implementations can include implementation in one or more computer programs that are executable and/or interpretable on a programmable system including at least one programmable processor, which may be special or general purpose, coupled to receive data and instructions from, and to transmit data and instructions to, a storage system, at least one input device, and at least one output device.

These computer programs (also known as programs, software, software applications or code) include machine instructions for a programmable processor, and can be implemented in a high-level procedural and/or object-oriented programming language, and/or in assembly/machine language. As used herein, the terms machine-readable medium and computer-readable medium refer to any computer program product, apparatus and/or device (e.g., magnetic discs, optical disks, memory, Programmable Logic Devices (PLDs)) used to provide machine instructions and/or data to a programmable processor, including a machine-readable medium that receives machine instructions as a machine-readable signal. The term machine-readable signal refers to any signal used to provide machine instructions and/or data to a programmable processor.

To provide for interaction with a user, the systems and techniques described here can be implemented on a computer having a display device (e.g., a CRT (cathode ray tube) or LCD (liquid crystal display) monitor) for displaying information to the user and a keyboard and a pointing device (e.g., a mouse or a trackball) by which the user can provide input to the computer. Other kinds of devices can be used to provide for interaction with a user as well; for example, feedback provided to the user can be any form of sensory feedback (e.g., visual feedback, auditory feedback, or tactile feedback); and input from the user can be received in any form, including acoustic, speech, or tactile input.

The systems and methods described herein can be implemented in a computing system that includes a back end component (e.g., as a data server), or that includes a middleware component (e.g., an application server), or that includes a front end component (e.g., a client computer having a graphical user interface or a Web browser through which a user can interact with an implementation of the systems and techniques described here), or any combination of such back end, middleware, or front end components. The components of the system can be interconnected by any form or medium of digital data communication (e.g., a communication network). Examples of communication networks include a local area network (LAN), a wide area network (WAN), and the Internet.

The computing system can include clients and servers. A client and server are generally remote from each other and typically interact through a communication network. The relationship of client and server arises by virtue of computer programs running on the respective computers and having a client-server relationship to each other.

### EXAMPLES

The following Example is merely illustrative and is not intended to limit the scope or content of the invention in any way.

### Example 1

### Fiber Device

An optical fiber device was constructed with three main components.

First, the buffer coating was stripped from a hydrogen-loaded photosensitive Corning SMF-28 fiber. Next, a chirped grating was inscribed across 10 mm of the stripped fiber with a pulsed KrF excimer laser (Light Machinery) by using the phase mask method (Laser & Photonics Reviews. 7(1): 83-108, 2013). The resulting chirped grating reflected light from 1551-1554 nm. The diameter of the fiber core was 8.2 um and the diameter of the entire fiber (core plus cladding) was 125 μm.

Second, the buffer coating was stripped from a hydrogen-loaded 2 dB/cm attenuating fiber (Coractive). In a single-pass configuration, 10 mm of this light-absorbing fiber (LAF) absorbed approximately 37% of incoming light and converted it into heat. In a double-pass configuration (i.e., with a chirped grating that reflects the incoming light back into the LAF), 10 mm of the LAF absorbed approximately 60% of the incoming and reflected light and converted it into heat. The diameter of the LAF core was approximately 7.5 μm. A Fiber Bragg Grating (FBG) was inscribed across the 10 mm length of the LAF by the same method as the chirped grating described above. The Bragg peak was centered at 1567 nm.

Third, a 5 mm-long section of low-loss multimode fiber (MMF) (Thorlabs) was spliced with a fusion splicer (Fujikura) in between two sections of Corning SMF-28 fiber. The MMF and SMF segments were stripped of their buffer coating. The diameter of the MMF core was 62.5 um. About 20% of light entering the MMF core was transmitted to the cladding of the spliced Corning SMF-28 fiber, and the other 80% was transmitted into the core.

These three components were cleaved and spliced together with a fusion splicer (Vytran), in the following order: MMF fiber spliced in between two sections of Corning SMF-28 fiber; LAF with inscribed FBG; and the chirped grating.

The resulting optical fiber device was about 40 mm in length, of which 10 mm was the length of the LAF with inscribed FBG. The device was then spliced onto a connectorized patchcord and connected through a mating sleeve to an optical setup.

Optical Setup

A bench-top optical setup was assembled to multiplex three different light sources and an optical spectrum analyzer into the core of the Corning SMF-28 fiber that was spliced upstream of the MMF. The three light sources were: (1) a 2 mW C+L (1525 nm-1610 nm) fiber coupled broadband source (JDS Uniphase) for interrogating the FBG; (2) a variable 0-2 W fiber coupled erbium-doped fiber amplifier (EDFA) (Amonics) seeded with a 2 mW fiber coupled tunable fiber laser (Hewlett Packard) adjusted to 1552.5 nm for providing the infrared (IR) light to be absorbed by the LAF and also reflected by the chirped grating; and (3) a 20 mW argon ion laser (JDS Uniphase) fiber-coupled after going through an optical shutter and a filter transmitting only the 488 nm line for fluorescent excitation of the DNA spots on the LAF surface (described below). These three sources, also coupled with the input of an optical spectrum analyser (OSA) (Ando), were multiplexed together using a combination of three fused optical fiber couplers (Thorlabs). The OSA allowed monitoring of the Bragg peak of the FBG.

In addition to this bench-top optical setup, an imaging system was assembled to monitor fluorescence of the DNA spots. The optical setup consisted of a camera (The Imaging Source), lenses, and a 515 nm-540 nm emission filter.

Control System

The EDFA IR pump, the optical shutter, the OSA wavelength acquisition, and the camera image acquisition were controlled by a computer to enable PCR thermal cycling and fluorescent detection. Specifically, the Bragg peak from the FBG was constantly monitored with the OSA, and this allowed real-time temperature measurement. The real-time temperature measurements enabled proportional-integral-derivative (PID) control of the EDFA pump power for thermal cycling. The optical shutter for the 488 nm source and the camera image acquisition were activated once or twice per thermal cycle during the PCR annealing phase. This enabled real-time fluorescence monitoring of the DNA spots on the LAF surface.

DNA Spots

PCR primers were designed to amplify a section of the ABCB1 gene and were synthesized by Integrated DNA Technologies. The sequence of the forward primer was: 5′-GAACATTGCCTATGGAGACA-3′ (SEQ ID NO: 1).

The sequence of the reverse primer was: 5′-CCAGGCTGTTTATTTGAAGA-3′ (SEQ ID NO: 2). The sequence of a reverse primer with a spacer/UV tag was 5′-TTTTTTTTTTCCCCCCCCCC-CCAGGCTGTTTATTTGAAGAGAGACTTACATT-3′ (SEQ ID NO: 3) (i.e., the sequence of the spacer/UV tag was 5′-TTTTTTTTTTCCCCCCCCCC-3′ (SEQ ID NO: 4)).

The reverse primers with spacer/UV tag were dissolved in phosphate buffered saline (PBS) to a concentration of 25 μM. Three drops of this solution were deposited along the LAF section of the fiber device to form three separate spots. Each drop contained approximately 1-2 μl of dissolved primer. The spotted fiber device was then dried in an oven at 60° C. It was then treated in a UV crosslinker (Thermofisher Scientific) with which was applied 0.3 Joules/cm² at a wavelength of 254 nm such as to attach the spacers/UV tag to the surface of the LAF. The dried and treated fiber device was then rinsed first with a solution of 0.1% Sodium Dodecyl Sulfate/0.1× Saline Sodium Citrate, and second with deionized water. The resulting spots were about 1-1.5 mm wide, and were spaced about 1-1.5 mm apart.

Reaction Vessel

The spotted fiber device was inserted into an open-ended glass capillary (50 mm length, 1.5 mm outer diameter, and 1.1 mm inner diameter) and centered transversally within the capillary using a v-groove attached to a 3D positioner (Thorlabs). The capillary was filled with 40 μl of PCR master mix. This master mix consisted of: 1.1× Colourless Buffer (Promega, Cat. No. M7921), 0.2 mM dNTPs (Enzymatics, Cat. No. N2050L), 1 mM MgCl2, 2.5 μg/μl BSA (Sigma, Cat. No. A9418), 0.5× SYBR Green (ThermoFisher, Cat. No. S7563), 0.5 μM of forward primer, 0.0625 μM of reverse primer, 3.88 Units of Hotstart Taq polymerase (Promega, Cat. No.M5001), and 5 nM of synthetic DNA template (Integrated DNA Technologies). The ends of the capillary were closed with mineral oil to prevent evaporation during PCR.

The sequence of the synthetic template was 5′-GAACATTGCCTATGGAGACAACAGCCGGGTGGTGTCACAGGAAGAGATCGTGAGGG CAGCAAAGGAGGCCAACATACATGCCTTCATCGAGTCACTGCCTAATGTAAGTCTCT CTTCAAATAAACAGCCTGG-3′ (SEQ ID NO: 5).

Thermal Cycling Conditions

The fiber device with glass capillary and PCR master mix underwent the following two-temperature thermal cycling program: 95° C. for 5 minutes for initial denaturation, followed by 15 cycles of 95° C. for 5 seconds and 56° C. for 60 seconds. Following thermal cycling, the temperature was held at 56° C. for 10 minutes to allow further extension and hybridization. Finally, the temperature was raised up to 95° C. in approximately 2 minutes while images were constantly acquired to extract data for calculating the melt curve.

Results

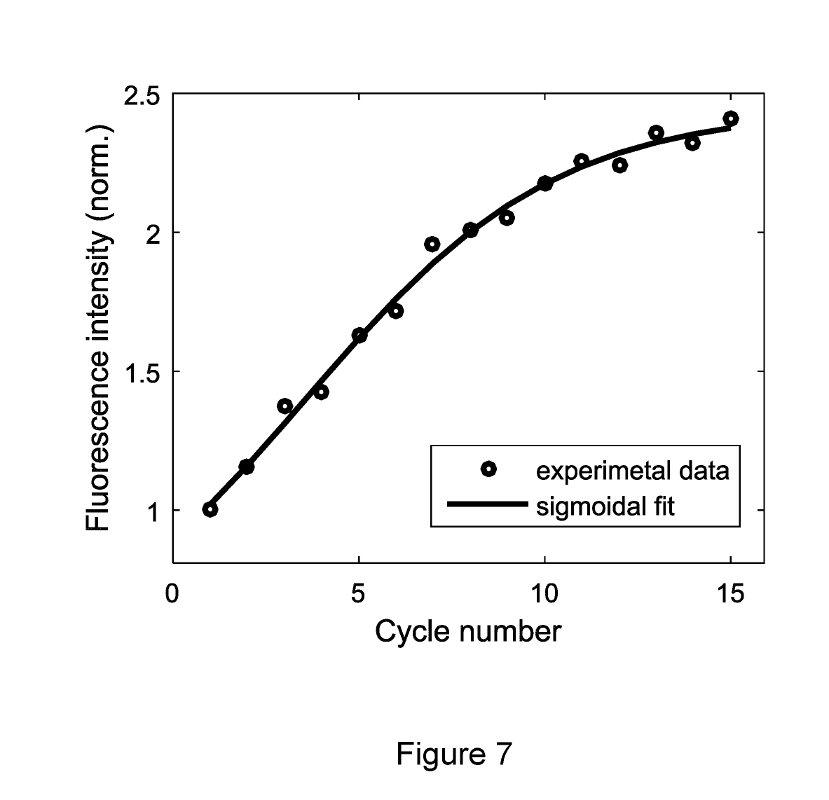

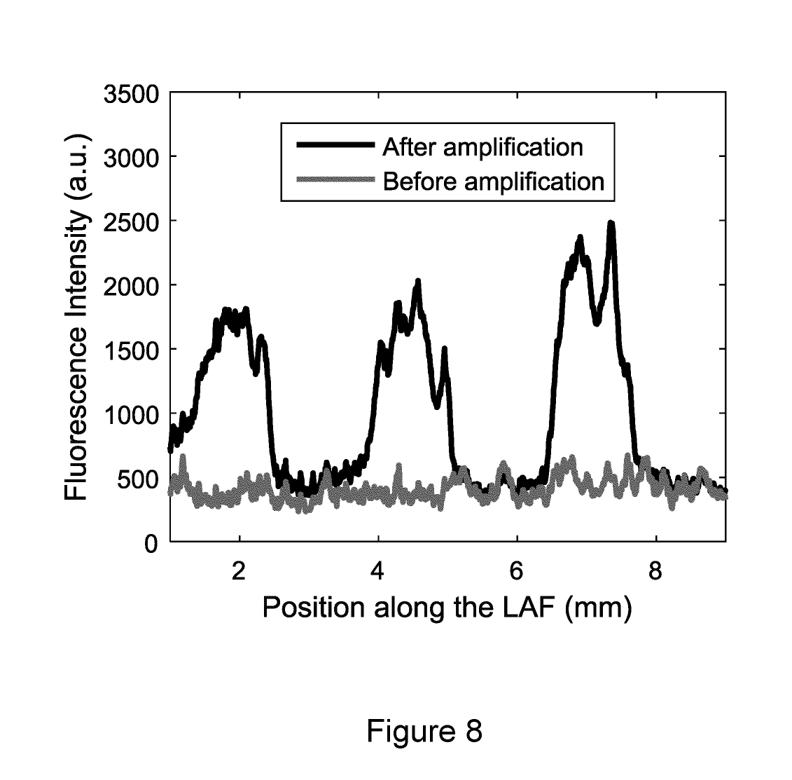

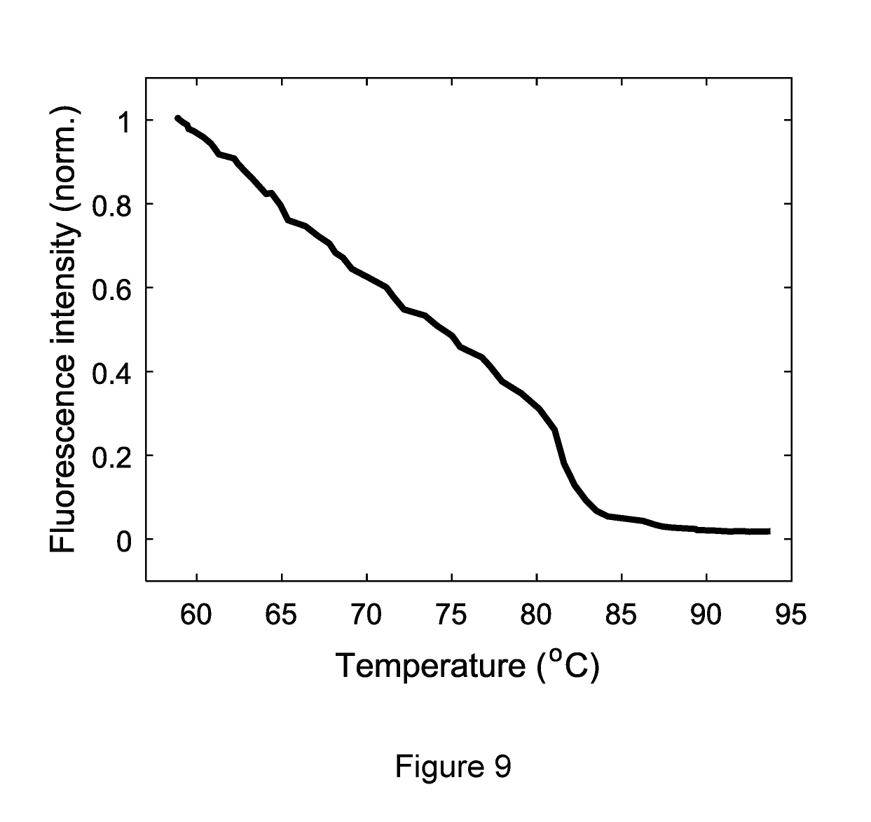

Unambiguous PCR amplification of the three spots is observed (FIGS. 7 and 8) and the melt curve confirms the specificity of the amplification (FIG. 9).

### Embodiments

1. A system comprising:

a light-absorbing optical fiber (LAF), which includes a first region and a second region, wherein the first region absorbs light at a first wavelength and the second region transmits light at a second wavelength and wherein the first and second regions both extend along the entire length of the LAF and each have longitudinal axes that are parallel to a longitudinal axis through the center of the LAF;

a first light source that produces light having the first wavelength and a second light source that produces light having the second wavelength; and

an optical coupling element configured to (i) couple light having the first wavelength from the first light source into the first region of the LAF and (ii) couple light having the second wavelength from the second light source into the second region of the LAF.
2. The system of embodiment 1, wherein the first wavelength is in the infrared region, e.g., from about 750 nm to 50 μm (e.g., from about 1300 to 1700 nm, e.g., from about 1552 to 1553 nm).
3. The system of embodiment 1 or 2, wherein the first region absorbs light at the first wavelength with an attenuation in the range of about 0.1 dB/cm to 10 dB/cm (e.g., in the range of about 1.5 dB/cm to 2.5 dB/cm).
4. The system of any one of embodiments 1 to 3, wherein the first region includes a dopant selected from the group consisting of the transition elements Co, Fe, Ni, Cr, Cu, Ti, Mn, V, and combinations thereof.
5. The system of any one of embodiments 1 to 4, wherein the second wavelength is in the region from about 390 nm to 700 nm (e.g., from about 450 nm to 500 nm, e.g., about 488 nm).
6. The system of any one of embodiments 1 to 5, wherein the first and second regions are in physical contact (e.g., wherein first region is a core region of the LAF and the second region is a cladding layer surrounding the core region of the LAF).
7. The system of any one of embodiments 1 to 6, wherein the LAF is about 2 mm to 50 mm in length (e.g., about 5 mm to 20 mm in length).
8. The system of any one of embodiments 1 to 7, wherein the first region is a core region of the LAF and the second region is a cladding layer surrounding the core region of the LAF, and wherein the diameter of the core region of the LAF is in the range of about 6 μm to 1000 μm (e.g., about 7μm to 50 μm).
9. The system of any one of embodiments 1 to 8, wherein power output of the first light source at the first wavelength is variable.
10. The system of embodiment 9, wherein the first light source is a variable amplifier (e.g., erbium-doped fiber amplifier (EDFA) seeded with a tunable fiber laser adjusted to the first wavelength).
11. The system of embodiment 9, wherein the first light source is a high power laser diode tuned to the first wavelength and controlled with a laser diode driver which has an adjustable current output.
12. The system of any one of embodiments 9 to 11, wherein power output of the first light source at the first wavelength can be varied from 0 Watt up to about 20 Watt (e.g., from 0 Watt up to about 2 Watt).
13. The system of any one of embodiments 1 to 12, wherein the second light source includes a laser upstream of an optical shutter and a filter that transmits only the second wavelength.
14. The system of any one of embodiments 1 to 12, wherein the second light source includes a fiber pigtailed laser diode with an output centered at the second wavelength.
15. The system of embodiment 14, wherein a laser diode driver is used to enable/disable the output of the fiber pigtailed laser diode.
16. The system of any one of embodiments 1 to 15, wherein the optical coupling element comprises a free space coupler.
17. The system of embodiment 16, wherein the system comprises an optical fiber element upstream of the free space coupler in which the first and second light sources are multiplexed.
18. The system of any one of embodiments 1 to 17, including a low-loss optical fiber which comprises a core region and a cladding layer surrounding the core region, and wherein the first and second wavelengths are multiplexed in the core region of the low-loss optical fiber.
19. The system of any one of embodiments 1 to 15, wherein the optical coupling element comprises a multimode interference (MMI) element spliced upstream of the LAF.
20. The system of embodiment 19, wherein the MMI element comprises a low-loss single mode optical fiber element spliced upstream of a low-loss multimode optical fiber element, and wherein the first and second wavelengths are multiplexed in the core of the low-loss single mode optical fiber element.
21. The system of embodiment 20, wherein the core of the low-loss multimode optical fiber is larger than the core of the LAF.
22. The system of any one of embodiments 1 to 21, wherein the LAF-based device comprises a low-loss optical fiber element spliced upstream of the LAF.
23. The system of embodiment 22, wherein the optical fiber element is a low-loss optical fiber which comprises a core region and a cladding layer surrounding the core region (e.g., a single cladding low-loss optical fiber, e.g., a double cladding low-loss optical fiber).
24. The system of embodiment 23, wherein the first wavelength is transmitted through the core region of the low-loss optical fiber and the second wavelength is transmitted through a cladding layer of the low-loss optical fiber.
25. The system of any one of embodiments 1 to 24, further comprising a reflective element (e.g., a mirror, e.g., a gold mirror, e.g., a chirped fiber grating, e.g., a chirped fiber grating which reflects light in the range from about 1551 nm to 1554 nm) located downstream of the LAF, wherein at least a portion of the light at the first wavelength is reflected back into the LAF by the reflective element (e.g., wherein the reflective element has a reflectivity greater than 80%, e.g., 90%, e.g., 95%) (e.g., wherein the combination of the first light pass and the reflected pass results in a relative difference in absorbed power (per unit length) between the downstream and upstream ends (e.g., a relative difference below 50%) and results in a total attenuation in the core of the LAF ranging from about 0.5 dB to 5.5 dB).
26. The system of embodiment 25, wherein the reflective element is a chirped fiber grating that has a reflection spectrum that both transmits the first wavelength at an annealing and/or extension temperature and reflects the first wavelength at a denaturation temperature.
27. The system of any one of embodiments 1 to 26, further comprising a Fiber Bragg Grating (FBG) inscribed within the first region of the LAF or within a low-loss optical fiber spliced upstream of the LAF.
28. The system of embodiment 27, further comprising a third light source that produces light covering a range of wavelengths (e.g., in the infrared region) for interrogating the FBG and an optical spectrum analyzer for monitoring the Bragg peak of the FBG.
29. The system of embodiment 27, further comprising a third light source that produces light having a third wavelength; and a power meter (or photodiode) with a bandpass filter for monitoring the reflected power of this third light source to infer the spectral position of the Bragg peak.
30. The system of embodiment 27, wherein the light produced by the third light source is in the infrared region, e.g., from about 750 nm to 3 μm (e.g., from about 1525 nm to 1610 nm, e.g., about 1567 nm).
31. The system of any one of embodiments 28, 29, or 30, wherein the third light source is a broadband infrared light source.
32. The system of embodiment 31, wherein power output of the third light source at the third wavelength or range of wavelengths is less than about 0.1 Watt (e.g., less than about 0.01 Watt).
33. The system of any one of embodiments 1 to 32, further comprising a detection element (e.g., an imaging element) for detecting (e.g., imaging) fluorescence on an outside surface of the second region of the LAF (e.g., a camera, e.g., a camera with lenses and an emission filter, e.g., a 515 nm to 540 nm emission filter).
34. The system of any one of embodiments 1 to 33, further comprising a support structure (e.g., a ferrule) in contact with the LAF or upstream low-loss fiber section.
35. The system of any one of embodiments 1 to 34, further comprising a reaction vessel, wherein at least a portion of the LAF is located within the reaction vessel (e.g., an open ended glass capillary).
36. The system of embodiment 35, wherein the reaction vessel includes a glass capillary closed on one side by a ferrule containing the LAF and closed on the other side by a cap.
37. The system of any one of embodiments 35 or 36, further comprising an immobilized (e.g., non-covalently, e.g., covalently) capture probe for an analyte on an outside surface of the second region of the LAF.
38. The system of embodiment 37, wherein the immobilized capture probe comprises a biomolecule selected from the group consisting of polynucleotides (e.g., RNA, DNA, PNA, oligonucleotide that are at least partially complementary to the analyte), polypeptides (e.g., antibodies or receptors that bind the analyte), and polysaccharides (e.g., that are bound by the analyte).
39. The system of embodiment 38, wherein a plurality of one or more types of capture probes specific for one or more types of analytes is immobilized in an array format on an outside surface of the second region of the LAF.
40. The system of any one of embodiments 37 to 39, further comprising a nanoparticle coating on an outside surface of the second region of the LAF.
41. The system of any one of embodiments 37 to 40, comprising an external heating and/or cooling element (e.g., wherein the element includes a member selected from the group consisting of a fan, an element that includes a liquid coolant, cooled and/or heated air streams, a Peltier module, a resistive heater, and combinations thereof).
42. A method comprising:

providing a system of any one of embodiments 35 to 41, wherein the reaction vessel includes a liquid sample; and transmitting light having the first wavelength from the first light source into the first region of the LAF to heat the liquid sample.
43. The method of embodiment 42, further comprising transmitting light having the second wavelength from the second light source into the second region of the LAF.
44. The method of embodiment 42 or 43, wherein a Fiber Bragg Grating (FBG) is inscribed within the first region of the LAF or within a low-loss optical fiber spliced upstream of the LAF and the system comprises a third light source that produces light for interrogating the FBG and an optical spectrum analyzer for monitoring the Bragg peak of the FBG, the method further comprising transmitting light from the third light source; and monitoring the Bragg peak of the FBG using the optical spectrum analyzer.
45. The method of any one of embodiments 42 to 44, further comprising detecting (e.g., imaging) (e.g., via a detection element (e.g., an imaging element)) fluorescence on an outside surface of the second region of the LAF (e.g., using a camera, e.g., a camera with lenses and an emission filter, e.g., a 515 nm to 540 nm emission filter), wherein the fluorescence is indicative of the presence of an analyte in the liquid sample.
46. The method of any one of embodiments 42 to 45, wherein the liquid sample comprises a nucleic acid analyte and amplification reagents (e.g., forward and reverse primers that hybridize to the nucleic acid analyte and its complement and a DNA polymerase) (e.g., wherein the liquid sample is a biological sample).
47. The method of embodiment 46, wherein forward and reverse primers are used with non-equal concentrations (e.g., at a ratio of from about 1:2 to about 1:4, e.g., at a ratio of about 1:3, e.g., ata ratio of from about 1:8 to about 1:12).
48. The method of any one of embodiments 42 to 47, further comprising an immobilized (e.g., non-covalently, e.g., covalently) capture probe for an analyte on an outside surface of the second region of the LAF (e.g., wherein the immobilized capture probe is a forward or reverse primer for amplification of a nucleic acid analyte, e.g., wherein the immobilized capture probe is an oligonucleotide comprising a section complementary to at least a portion of the analyte).
49. The method of any one of embodiments 42 to 48, wherein the immobilized capture probe comprises an oligonucleotide that hybridizes to a nucleic acid analyte.
50. The method of any one of embodiments 42 to 49, wherein the liquid sample comprises a nucleic acid analyte.
51. The method of embodiment 50, wherein the liquid sample further comprises a fluorescent reporter that preferentially binds to double-stranded nucleic acid molecules over single-stranded nucleic acid molecules (e.g., double-stranded DNA over single-stranded DNA) and absorbs light at the second wavelength (e.g., an intercalating dye, e.g., SYBR green).
52. The method of embodiment 50, wherein the liquid sample further comprises a labeled (e.g., with a fluorophore) oligonucleotide detection probe that directly or indirectly hybridizes to the nucleic acid analyte.
53. The method of embodiment 50, wherein the oligonucleotide detection probe is a molecular beacon detection probe.
54. The method of embodiment 53, wherein a plurality of different molecular beacons are used to detect a plurality of different nucleic acid analytes in the liquid sample.
55. The method of any one of embodiments 42 to 54, further comprising adjusting the output power of the first light source (e.g., in the range of 0 Watt up to about 20 Watt, e.g., in the range from 0 Watt up to about 2 Watt) to cycle a temperature of the liquid sample.
56. The method of embodiment 55, wherein the step of adjusting leads to PCR amplification of a nucleic acid analyte in the liquid sample.
57. The method of embodiment 56, wherein the PCR amplification involves extension of at least one forward and/or reverse primer that is immobilized on the surface of the second region of the LAF.
58. The method of any one of embodiments 55 to 57, wherein the step of adjusting increases the temperature of the liquid sample.
59. The method of any one of embodiments 55 to 58, wherein the step of adjusting reduces the temperature of the liquid sample.
60. The method of any one of embodiments 42 to 54, further comprising controlling the output power of the first light source to maintain a temperature of the liquid sample.
61. The method of embodiment 60, wherein the step of controlling leads to isothermal amplification of a nucleic acid analyte in the liquid sample.
62. The method of embodiment 61, wherein the isothermal amplification involves extension of at least one forward and/or reverse primer that is immobilized on the surface of the second region of the LAF.
63. The method of any one of embodiments 42 to 62, comprising heating and/or cooling the system via a heating and/or cooling element (e.g., wherein the heating and/or cooling element includes a member selected from the group consisting of a fan, an element that includes a liquid coolant, cooled and/or heated air streams, a Peltier module, a resistive heater, and combinations thereof) (e.g., wherein the heating and/or cooling is achieved by using a combination of the heating and/or cooling elements and IR light).
64. The method of any one of embodiments 42 to 63, comprising monitoring temperature via a member selected from the group consisting of a thermistor, a thermocouple, an RTD, and a non-contact IR thermometer (e.g., wherein the temperature is also monitored by the system of any one of embodiments 35 to 41).
65. The method of any one of embodiments 42 to 64, wherein an external (not fiber-coupled) light source is used (e.g., to monitor fluorescence in the liquid sample).

We claim:

1. A system comprising:
a light-absorbing optical fiber (LAF), which includes a first region and a second region, wherein the first region absorbs light at a first wavelength and the second region transmits light at a second wavelength and wherein the first and second regions both extend along the entire length of the LAF and each have longitudinal axes that are parallel to a longitudinal axis through the center of the LAF;
a first light source that produces light having the first wavelength and a second light source that produces light having the second wavelength; and
an optical coupling element configured to (i) couple light having the first wavelength from the first light source into the first region of the LAF and (ii) couple light having the second wavelength from the second light source into the second region of the LAF.

2. The system of claim 1, wherein the first wavelength is in the infrared region.

3. The system of claim 1 or 2, wherein the first region absorbs light at the first wavelength with an attenuation in the range of about 0.1 dB/cm to 10 dB/cm.

4. The system of any one of claims 1 to 3, wherein the first region includes a dopant selected from the group consisting of the transition elements Co, Fe, Ni, Cr, Cu, Ti, Mn, V, and combinations thereof.

5. The system of any one of claims 1 to 4, wherein the second wavelength is in the region from about 390 nm to 700 nm.

6. The system of any one of claims 1 to 5, wherein the first and second regions are in physical contact.

7. The system of any one of claims 1 to 6, wherein the LAF is about 2 mm to 50 mm in length.

8. The system of any one of claims 1 to 7, wherein the first region is a core region of the LAF and the second region is a cladding layer surrounding the core region of the LAF, and wherein the diameter of the core region of the LAF is in the range of about 6 μm to 1000 μm.

9. The system of any one of claims 1 to 8, wherein power output of the first light source at the first wavelength is variable.

10. The system of claim 9, wherein the first light source is a variable amplifier.

11. The system of claim 9, wherein the first light source is a high power laser diode tuned to the first wavelength and controlled with a laser diode driver which has an adjustable current output.

12. The system of any one of claims 9 to 11, wherein power output of the first light source at the first wavelength can be varied from 0 Watt up to about 20 Watt.

13. The system of any one of claims 1 to 12, wherein the second light source includes a laser upstream of an optical shutter and a filter that transmits only the second wavelength.

14. The system of any one of claims 1 to 12, wherein the second light source includes a fiber pigtailed laser diode with an output centered at the second wavelength.

15. The system of claim 14, wherein a laser diode driver is used to enable/disable the output of the fiber pigtailed laser diode.

16. The system of any one of claims 1 to 15, wherein the optical coupling element comprises a free space coupler.

17. The system of claim 16, wherein the system comprises an optical fiber element upstream of the free space coupler in which the first and second light sources are multiplexed.

18. The system of any one of claims 1 to 17, including a low-loss optical fiber which comprises a core region and a cladding layer surrounding the core region, and wherein the first and second wavelengths are multiplexed in the core region of the low-loss optical fiber.

19. The system of any one of claims 1 to 15, wherein the optical coupling element comprises a multimode interference (MMI) element spliced upstream of the LAF.

20. The system of claim 19, wherein the MMI element comprises a low-loss single mode optical fiber element spliced upstream of a low-loss multimode optical fiber element, and wherein the first and second wavelengths are multiplexed in the core of the low-loss single mode optical fiber element.

21. The system of claim 20, wherein the core of the low-loss multimode optical fiber is larger than the core of the LAF.

22. The system of any one of claims 1 to 21, wherein the LAF-based device comprises a low-loss optical fiber element spliced upstream of the LAF.

23. The system of claim 22, wherein the optical fiber element is a low-loss optical fiber which comprises a core region and a cladding layer surrounding the core region.

24. The system of claim 23, wherein the first wavelength is transmitted through the core region of the low-loss optical fiber and the second wavelength is transmitted through a cladding layer of the low-loss optical fiber.

25. The system of any one of claims 1 to 24, further comprising a reflective element located downstream of the LAF, wherein at least a portion of the light at the first wavelength is reflected back into the LAF by the reflective element.

26. The system of claim 25, wherein the reflective element is a chirped fiber grating that has a reflection spectrum that both transmits the first wavelength at an annealing and/or extension temperature and reflects the first wavelength at a denaturation temperature.

27. The system of any one of claims 1 to 26, further comprising a Fiber Bragg Grating (FBG) inscribed within the first region of the LAF or within a low-loss optical fiber spliced upstream of the LAF.

28. The system of claim 27, further comprising a third light source that produces light covering a range of wavelengths for interrogating the FBG and an optical spectrum analyzer for monitoring the Bragg peak of the FBG.

29. The system of claim 27, further comprising a third light source that produces light having a third wavelength; and a power meter with a bandpass filter for monitoring the reflected power of this third light source to infer the spectral position of the Bragg peak.

30. The system of claim 27, wherein the light produced by the third light source is in the infrared region.

31. The system of any one of claim 28, 29, or 30, wherein the third light source is a broadband infrared light source.

32. The system of claim 31, wherein power output of the third light source at the third wavelength or range of wavelengths is less than about 0.1 Watt.

33. The system of any one of claims 1 to 32, further comprising a detection element for detecting fluorescence on an outside surface of the second region of the LAF.

34. The system of any one of claims 1 to 33, further comprising a support structure in contact with the LAF or upstream low-loss fiber section.

35. The system of any one of claims 1 to 34, further comprising a reaction vessel, wherein at least a portion of the LAF is located within the reaction vessel.

36. The system of claim 35, wherein the reaction vessel includes a glass capillary closed on one side by a ferrule containing the LAF and closed on the other side by a cap.

37. The system of any one of claim 35 or 36, further comprising an immobilized capture probe for an analyte on an outside surface of the second region of the LAF.

38. The system of claim 37, wherein the immobilized capture probe comprises a biomolecule selected from the group consisting of polynucleotides, polypeptides, and polysaccharides.

39. The system of claim 38, wherein a plurality of one or more types of capture probes specific for one or more types of analytes is immobilized in an array format on an outside surface of the second region of the LAF.

40. The system of any one of claims 37 to 39, further comprising a nanoparticle coating on an outside surface of the second region of the LAF.

41. The system of any one of claims 37 to 40, comprising an external heating and/or cooling element.

42. A method comprising:
providing a system of any one of claims 35 to 41, wherein the reaction vessel includes a liquid sample; and
transmitting light having the first wavelength from the first light source into the first region of the LAF to heat the liquid sample.

43. The method of claim 42, further comprising transmitting light having the second wavelength from the second light source into the second region of the LAF.

44. The method of claim 42 or 43, wherein a Fiber Bragg Grating (FBG) is inscribed within the first region of the LAF or within a low-loss optical fiber spliced upstream of the LAF and the system comprises a third light source that produces light for interrogating the FBG and an optical spectrum analyzer for monitoring the Bragg peak of the FBG, the method further comprising transmitting light from the third light source; and monitoring the Bragg peak of the FBG using the optical spectrum analyzer.

45. The method of any one of claims 42 to 44, further comprising detecting fluorescence on an outside surface of the second region of the LAF, wherein the fluorescence is indicative of the presence of an analyte in the liquid sample.

46. The method of any one of claims 42 to 45, wherein the liquid sample comprises a nucleic acid analyte and amplification reagents.

47. The method of claim 46, wherein forward and reverse primers are used with non-equal concentrations.

48. The method of any one of claims 42 to 47, further comprising an immobilized capture probe for an analyte on an outside surface of the second region of the LAF.

49. The method of any one of claims 42 to 48, wherein the immobilized capture probe comprises an oligonucleotide that hybridizes to a nucleic acid analyte.

50. The method of any one of claims 42 to 49, wherein the liquid sample comprises a nucleic acid analyte.

51. The method of claim 50, wherein the liquid sample further comprises a fluorescent reporter that preferentially binds to double-stranded nucleic acid molecules over single-stranded nucleic acid molecules and absorbs light at the second wavelength.

52. The method of claim 50, wherein the liquid sample further comprises a labeled oligonucleotide detection probe that directly or indirectly hybridizes to the nucleic acid analyte.

53. The method of claim 50, wherein the oligonucleotide detection probe is a molecular beacon detection probe.

54. The method of claim 53, wherein a plurality of different molecular beacons are used to detect a plurality of different nucleic acid analytes in the liquid sample.

55. The method of any one of claims 42 to 54, further comprising adjusting the output power of the first light source to cycle a temperature of the liquid sample.

56. The method of claim 55, wherein the step of adjusting leads to PCR amplification of a nucleic acid analyte in the liquid sample.

57. The method of claim 56, wherein the PCR amplification involves extension of at least one forward and/or reverse primer that is immobilized on the surface of the second region of the LAF.

58. The method of any one of claims 55 to 57, wherein the step of adjusting increases the temperature of the liquid sample.

59. The method of any one of claims 55 to 58, wherein the step of adjusting reduces the temperature of the liquid sample.

60. The method of any one of claims 42 to 54, further comprising controlling the output power of the first light source to maintain a temperature of the liquid sample.

61. The method of claim 60, wherein the step of controlling leads to isothermal amplification of a nucleic acid analyte in the liquid sample.

62. The method of claim 61, wherein the isothermal amplification involves extension of at least one forward and/or reverse primer that is immobilized on the surface of the second region of the LAF.

63. The method of any one of claims 42 to 62, comprising heating and/or cooling the system via a heating and/or cooling element.

64. The method of any one of claims 42 to 63, comprising monitoring temperature via a member selected from the group consisting of a thermistor, a thermocouple, an RTD, and a non-contact IR thermometer.

65. The method of any one of claims 42 to 64, wherein an external light source is used.
